(19) United States
(12) Patent Application Publication
(10) Pub. No.: US 2026/0354804 A1
(43) Pub. Date: Nov. 12, 2026

(54) A DEVICE FOR SKIN BIOPSY AND SCAR REVISION

(71) Applicants: Dermcut Medical Pte. Ltd., Singapore (SG); Singapore Health Services Pte. Ltd., Singapore (SG)

(72) Inventors: Wei Jian Banjamin QUEK, Singapore (SG); Avshalom SHENHAV, Singapore (SG); Chi Yuan Ian LOH, Singapore (SG)

(21) Appl. No.: 19/515,509

(22) PCT Filed: Aug. 30, 2024
(86) PCT No.: PCT/SG2024/050558

§ 371 Date: Mar. 2, 2026

(30) Foreign Application Priority Data
Aug. 31, 2023 (SG) 10202302455X

(51) Int. Cl. A61B 10/02; A61B 17/00; A61B 17/3209

(52) U.S. Cl. CPC A61B 10/0233; A61B 17/32093; A61B 2010/0208; A61B 2017/00761

(57) ABSTRACT

Herein disclosed is a device operable to perforate a skin, the device comprising: a housing comprising an upper member and a bottom member, wherein the upper member is detachably engaged to the bottom member; an actuation mechanism housed proximal to the upper member; a biasing member operably coupled to the actuation mechanism; one or more striking members; and more than one blades, each of the more than one blades operably coupled to a corresponding striking member of the one or more striking members, wherein the biasing member is operable to have the actuation mechanism activate an actuating member to drive each of the one or more striking members in a manner which renders each of the more than one blades to perforate the skin.

### CROSS-REFERENCE TO RELATED APPLICATION

This application is a national phase of PCT/SG2024/050558 filed on 30 Aug. 2024, which claims the benefit of priority of Singapore Patent Application No. 10202302455X, filed 31 Aug. 2023, the content of both being hereby incorporated by reference in their entirety for all purposes.

### TECHNICAL FIELD

The present disclosure relates to a device operable to perforate a skin, such as in skin biopsy and scar revision.

### BACKGROUND

Traditionally, scapel and biopsy punches are used as cutting devices to remove sections of skin and revise scars. However, these devices, and the methods arising from use of such devices, tend to have their limitations.

For example, traditional devices for revising scars may be very tedious and time consuming to operate and/or achieve the desired results. In certain instances, for example, the use of traditional devices may require excision of scar by drawing and planning the angle of cuts, each to be very precise, therefore taking a considerable amount of time and skills to have every step done accurately. Good motor skills tend to be required to perform these cuts and minimize serrated edges, which may be especially so for W-plasty that tend to require stitching from one point to another and any flaps must fit in nicely like a puzzle to avoid undesirable scarring. A scar of 2 cm to 3 cm scar may often already take 1.5 hours to 2 hours long using such traditional devices. Moreover, the angle of cuts must often be aligned against the scar and relaxed skin tension lines (RSTLs), as drawing tends to be done manually on the skin and/or excised by scalpel. Further, there may be a potential for serrated edges to form on the skin, especially from surgeons using traditional devices with less experience.

Further limitations with traditional devices may include (i) the use of a considerable high amount of force just to make a cut over a large area which is undesirable, (ii) low blades angles and undesirable lengths that produce low cutting efficiency and hence requiring more force for excision, (iii) susceptibility to poor angles of the W-plasty stencil, (iv) poor or no mechanism of releasing any blades without reducing human errors, and (v) poor or no mechanism to stop the blades at the correct height to prevent cuts that are too deep.

There is thus a need to provide for a solution that addresses one or more of the limitations mentioned above. The solution should at least provide for a device which resolves one or more of aforesaid limitations in devices for skin biopsy and/or scar revision.

### SUMMARY

In a first aspect, there is provided a device operable to perforate a skin, the device comprising:
a housing comprising an upper member and a bottom member, wherein the upper member is detachably engaged to the bottom member;
an actuation mechanism housed proximal to the upper member;
a biasing member operably coupled to the actuation mechanism;
one or more striking members; and
more than one blades, each of the more than one blades operably coupled to a corresponding striking member of the one or more striking members,
wherein the biasing member is operable to have the actuation mechanism activate an actuating member to drive each of the one or more striking members in a manner which renders each of the more than one blades to perforate the skin in less than 5 seconds.

### BRIEF DESCRIPTION OF THE DRAWINGS

The drawings are not necessarily to scale, emphasis instead generally being placed upon illustrating the principles of the present disclosure. In the following description, various embodiments of the present disclosure are described with reference to the following drawings, in which:

FIG. 1 shows components of a device of the present disclosure. Top left image shows a “jig” with slits 1006 for forming a V-shaped cut pattern (i.e., V-plasty pattern). Top right image shows a “jig” cover with spring-loaded mechanism 1002 (actuation mechanism) housed therein, wherein the spring-loaded mechanism 1002 is not shown. The two jigs formed a housing, wherein the jig with slits 1006 and jig with cover may be referred to as bottom member 1004 and upper member 1000, respectively. Bottom left image illustrates a pin 1008 and bottom right image shows a discretized scalpel blade 1024. The pin 1008 acts as a biasing member, which one or more actuating members (e.g., one or more springs) may be bias against to have the spring in a compressed state.

FIG. 2 shows in the top left image how the bottom member 1004 and upper member 1000 of FIG. 1 can be configured to form the housing. The top left image also shows the pillars 1012 of the bottom member 1004, which serves as a guide for engaging (e.g., fitting) the upper member 1000 (also see FIG. 3). For example, the shape and dimensions of the upper member 1000 can be configured to fit into the pillars 1012 of the bottom member 1004. The base of the bottom member 1004 may be a filleted base as shown in the bottom left image. As shown in the right image, the upper member 1000 can have two opposing openings 1014, 1016 (e.g., both shaped as rectangular openings) for receiving, insertion, holding and removal of the pin 1008 shown in FIG. 1. The right image shows the upper member 1000 has a window 1020, which allows observation of the actuation mechanism (e.g., spring) for any troubleshooting. The right image shows striker holes 1018, which only allows respective strikers to pass through. The tunnel holes (not shown) at the bottom of the upper member 1000 allow head of respective strikers (i.e., striking members) and spring to enter the tunnel.

FIG. 3 shows the internal structure of the upper member 1000 of the device. The height of the upper member 1000 can be configured to accommodate the dimensions (e.g., length of the actuating member). For example, the actuating member can include one or more springs, wherein the one or more springs can be housed in the upper member 1000 that is configured to have the one or more springs with a 2 mm pre-compression. That is to say, the one or more springs housed in the upper member 1000 can be kept under a compressed state prior to use of the device. When fully loaded with the blades 1024 and pin 1008, the one or more springs may be compressed by about 16 mm, yielding a spring force of 16 N that is used to drive the blades 1024 into a skin when the pin 1008 is removed. The tunnels as shown within the upper member 1000 define the pathways for respective striking members and springs to move vertically (up and down) therein. Each tunnel is connected to a striker hole 1018.

FIG. 4 shows a non-limiting example of an actuating member 1022 used in the actuation mechanism. In this non-limiting example, the actuating member is a spring, which is coupled to a striking member. The spring can be wrapped around the striking member.

FIG. 5 shows the two different states of the actuation mechanism and the operation of the actuating members 1022 of FIG. 4 for the two states. Prior to operation, in the first state, the actuating member 1022 (e.g., the spring) and the striking member in this instance, are loaded in the housing and the pin 1008 maintains the spring in a compressed state with no contact to respective blades 1024 in the bottom member 1004 as shown in the left image. During use, the pin 1008 is removed to have the actuating member 1022 in the second state. As shown in the right image, after removing the pin 1008, the compressed springs (together with striking members) launch (e.g., with a force of 16 N) to strike the top of respective blades 1024, pushing the blades 1024 to puncture the skin.

FIG. 6 shows two images depicting how the pin 1008 is removed (e.g., pulled) from the device 10 to release the spring-loaded strikers (see FIG. 4 and FIG. 5) for striking multiple blades to produce the punch on the uneven skin surface 10000.

FIG. 7 shows two non-limiting examples of the striking members. In the left image, the striking member 1022a is a separate component from the blade 1024 and spring. The striking member 1022a is a component that hits the blade 1024 with the force actuated by the spring so that the blade 1024 are driven toward the skin for puncturing and cutting the skin. In the right image, the striking member and the blade are joined as a single piece to form the striking member 1026. The striking member 1026 may help in blade retention as it has a head (i.e., nut) distal from the blade to confine the blade within the device.

FIG. 8 shows in the left image a commercially available penknife blade while the right image shows a blade of the present disclosure. As can be seen, the blade of the present disclosure is dimensioned differently to improve cutting performance. Both blades include a main body 802 and an edge 804. The edge 804 extends from the main body 802 by about 2 mm to about 5 mm and/or has a length of about 7 mm to about 9 mm. In this instance, the edge of the blade in the left image extends from main body by about 5 mm and has a length of about 8.53 mm while the edge of blade in the right image extends from main body by about 2 mm and has a length of about 7 mm. Understandably, the length of the edge refers to the longest dimension of the edge, which is identified in this instance by the dimensions of 8.53 mm and 7 mm in FIG. 8.

FIG. 9 shows a table setting out the different parameters of the blade (the parameters help define the tip at edge of blade), wherein the bevel angle (tip angle), cutting and tip radius, between a commercially available (off-the-shelves) blade and a blade of the present disclosure are compared. Based on the parameters, it can be recognized that the present blade is considerably sharper and better meets clinical requirements at least in terms of sharpness.

FIG. 10 shows the different W-plasty patterns from different configurations of blades arranged in the device. The degree of 100, 110 and 120 refers to an angle of a crosshatch in the lower pattern. A crosshatch of 120° is defined in the lower pattern as shown in FIG. 11. The blades used in these instances have an edge of 9 mm.

FIG. 11 shows a desirable W-plasty pattern considered by medical practitioners amongst the patterns of FIG. 10. The W-plasty pattern has an upper pattern and a lower pattern. The tip of the “Vs” (denoted by the 90° at the upper and lower patterns) of the geometrical W-Plasty pattern are symmetrical and therefore, allows the surgeons to stitch the tip to tip together, forming a fine line which is significantly less visible. Moreover, excising the scar in this manner promotes better healing for the wound. The two crosshatches in the upper pattern are each defined by an angle of 85° and the two crosshatches in the lower pattern are each defined by an angle of 120°. The length of the W-plasty pattern is measured in a manner as shown in FIG. 11 denoted by the dimension of <3.5 mm. The “blade length” in this instance refers to the length of the edge 802 of the blade (see FIG. 8), which is 7 mm.

FIG. 12 is a table indicating the parameters of a spring used. As skin is viscoelastic, different parameters of the springs may have to be considered, such as spring constant, spring force, elastic spring energy, spring velocities upon impact and spring travel distance. For certain experiments, spring constant and spring travel distance were considered to determine the cutting prowess of the device. The “spring travel distance” in the context of the present disclosure refers to the maximum displacement that a spring can be compressed or stretched from its resting state (uncompressed or non-stretched state). It may be used as a measure of how deep the blades can perforate the skin when subject to an external force.

FIG. 13 shows the positioning of the striking members and blades for deriving the W-plasty patterns in FIG. 10 and FIG. 11. As seen from the positioning, there may be little space to accommodate 10 strikers in the device to avoid interference between neighboring strikers and springs. The diameter of each striker's head and striker's stud may be configured to deal with this, e.g., not more than 5 mm and not more than 2.5 mm, respectively. The striker's stud refers to the part of the striker that is configured to contact the blade. Also, the number of springs required for a 2 to 4 cm scar, as an example, may be 10 to 18 springs depending on the number of V-shapes required and based on this the total static frictional force to overcome to release the springs may be 162 N (about 16.5 kg of load). A high static friction renders difficulties for the user to release the springs. Mechanical leverage (a push-to-release instead of pull-to-release) was considered to reduce the amount of force required for user to release the springs in order to minimize the excessive movement of the device during its operation.

FIG. 14 shows components of a device of the present disclosure. The components can be configured for both push-to-release and pull-to-release actuation mechanisms. Starting anti-clockwise from top right image, an upper member 2000 with striker holes is shown. Next is an intermediate member 2002 for extending height of device. The bottom member 2004 for forming the W-plasty pattern is shown left to the intermediate member. The image (bottom left) shows a contact plate 2006. The contact plate 2006 may be a metal contact plate. Right of the contact plate 2006 is shown the biasing member 2008b (instead of a pin). The biasing member 2008b may be coupled to a grip element 2008a through which a user controls movement of the biasing member 2008b. The biasing member 2008b can include multiple moveable plates. The contact plate 2006 helps support the biasing member 2008b in that the contact plate 2006 itself does not warp easily (warping may cause the actuation mechanism and hence the device to fail), especially for metal contact plate. Understandably, the strength of the contact plate supports the biasing member 2008b (e.g., the moveable plates) from warping under the stress of a compressed spring used as the actuating member (not shown in FIG. 14). Moreover, the static friction coefficient between metallic surfaces (e.g., a metal contact plate) is much lower than the static friction coefficient between plastic surfaces, thus the metal contact plate 2006 helps reduce the amount of force required for a user to operate the biasing member 2008b. The blade 1024 and striker 1022a are also shown. The number of striker holes in upper member 2000 may correspond to the number of strikers 1022a used.

FIG. 15 shows the front (left image) and back (right image) views of the device assembled from components of FIG. 14. The length 20002, width 20000, and height 20004 of the device are shown, and may be 52 mm, 25 mm and 72 mm as non-limiting examples, respectively.

FIG. 16 illustrates how the push-to-release mechanism works (left image). The strikers 1022a are partially blocked by the biasing member (e.g., a moveable metal plate) 2008b. Upon pushing (denoted by the three arrows) the grip element 2008 a (not shown) slightly (e.g., about 2 mm), the strikers 1022a fall through the keyholes on the biasing member 2008b and also releasing the springs to produce the W-plasty cut. The grip element 2008a can be pushed all at the same time or individually, which confers more flexibility to a user in controlling how the cut pattern can be formed. For example, the biasing member 2008b can have keyholes configured in a manner that corresponds to the arrangement of the strikers 1022a, which in turn corresponds to a W-plasty pattern to be formed. The biasing member 2008b may comprise of three portions, such that 3 to 4 springs can be released at a time, to allow the user to push the grip element 2008a more easily facing lesser static friction. FIG. 16 also illustrates how the pull-to-release mechanism works (right image). The device based on pull-to-release mechanism can be configured using the same components of a device operable based on the push-to-release mechanism, except that the biasing member and grip elements are configured for a user to exercise a pull of the grip elements and hence the biasing member to release the spring. Other than moveable metal plates, moveable plates formed of plastic materials may also be used if the plastic is able to withstand and/or is free of (e.g., has sufficient strength) flexing or warping issues. The grip element can be formed of metal or plastic. Compared to the push-to-release mechanism, the user operating the device configured with a pull-to-release mechanism may have to move (pull) the grip element for a distance of 20 mm to release the spring, which may be susceptible to introducing jerky motion when operating the device.

FIG. 17 shows components of a device of the present disclosure, wherein the device is modified to include a blade retention mechanism/component and a blade depth control mechanism/component. The blade retention mechanism/component helps retain the blade in the device (mitigate risk of blades falling off from the device so as to avoid harm to a user and patient). The blade retention mechanism/component and/or the blade depth control mechanism/component also help prevent and/or stop the blades from penetrating too deep into skin, as over-penetration may damage deep tissues and nerves. In other words, the device is configurable to have an improved depth control of the blades. The components include an upper member 4000, a bottom member 4004, an intermediate member 4002, an elevation member 4028, a top cover 4026, a contact plate 4006 (e.g., a metal contact plate), a biasing member 4008b, grip element 4008a and the striking member 1026 (with the striker's stud joined to a blade).

FIG. 18 shows the front (left image) and back (right image) views of the device assembled from components of FIG. 17. The length, width, and height of the device may be 62 mm, 32 mm and 98 mm as non-limiting examples, respectively.

FIG. 19 compares two devices having the push-to-release actuation mechanism (also for pull-to-release actuation mechanism) configured at different positions (demarcated with an ellipse). The actuation mechanism in the right image may be configured if more space is needed to increase the number or size of components, which may help improve lifetime and reliability of the mechanism.

FIG. 20 shows the position of the striker cap 1026a (i.e., striker head). The height 40000a conferred by the elevation member 4028 affects the plunging distance of the strikers 1026, understandably acting as the blade depth control mechanism/component. In various non-limiting instances, the motion of the plunging strikers 1026 are stopped at a surface of the top portion (i.e., the top surface) of the upper member 4000 which the striker cap 1026a contacts, understandably acting as the blade retention mechanism/component. The top surface of upper member 4000 is the surface which contacts the elevation member 4028. The height of the elevation member 4028 controls the distance which the striker falls through before it is stopped by the top surface of upper member 4000. The device can have an additional blade retention mechanism in place to prevent the blade from perforating the skin too deeply (e.g., more than 8 mm). For example, the stud of the striker 1026 can come into contact with a surface of a bottom portion (i.e., the bottom surface) of the upper member 4000, which may reside in the bottom member 4002 (e.g., the bottom surface of the upper member 4000 may be housed between the top surface and the bottom surface of the bottom member 4002, also see FIG. 18). By varying the height of the upper member 4002 which the striker falls through, the depth at which the blade can perforate into the skin may be effectively controlled.

FIG. 21 shows different keyholes configuration of the biasing member (e.g., moveable plates). The left image shows keyholes configured to accommodate strikers falling through the keyholes (see right image in FIG. 19) while the left image shows keyholes configured to accommodate strikers release at top of device (see right image in FIG. 19). In any case, the operating principles remain the same, i.e., to have the actuation mechanism release the spring so as to drive the strikers to hit the blades.

FIG. 22 shows the lock (left image) and unlocked (right image) position of the actuating members of the actuation mechanisms with respect to the strikers.

FIG. 23 shows different bottom member (see bottom member 4004 in FIG. 17) configured with different shapes, wherein the right image shows a less bulky bottom member which improves visibility of the scar when the bottom member is placed on skin.

FIG. 24 shows different bottom member sizes for handling different scar lengths, e.g., 3 cm and 4 cm scars, which are statistically median scar lengths. The bottom member for 3 cm and 4 cm scar revisions may be 72 mm and 82 mm in length, respectively.

FIG. 25 is a diagram showing certain parameters developed for a device of the present disclosure. The device is illustrated in more details, as non-limiting examples, in FIG. 26 to FIG. 43.

FIG. 26 shows the front view (left image) and a perspective view (right image) of a device of the present disclosure.

FIG. 27 shows a W-plasty cut pattern for a 2 cm scar to be exercise on by the present device. FIG. 27 also shows how a scar width and scar length is measured. The angle defining each of the two crosshatches of the lower pattern is 115°. The angle defining the crosshatches of the upper pattern in this instance is 70°. In various instances, the angle defining the crosshatches of the upper pattern may be in the range of 65° to 90° (e.g., 70° to 85°).

FIG. 28 shows components of a device shown in FIG. 26, wherein the device is modified to include a safety pin. The components include an upper member 5000, a bottom member 5004, an intermediate member 5002, an elevation member 5028, a top cover 5026, a contact plate 5006 (e.g., a metal contact plate), a biasing member 5008b, grip element 5008a and the striking members (with the striker's stud joined to a blade) 5202, 5204, 5206, and a spring 5300.

FIG. 29 shows the blade design for the device of FIG. 26 to FIG. 28. It can be seen that the blade has a crescent shape (see left image), wherein the blade tip concaves inward (i.e., away from the surface which the blade tip comes into contact with). Also, the blade tip denoted “A” in the center image, has a cutting angle of about 22° (see right image).

FIG. 30 shows the configuration and details of a striking member (joined to a blade).

FIG. 31 shows from a bottom-up view of the blade arrangement in the device. Specifically, the angles between two blades include 70, 115 and a single blade. The cut pattern corresponding to such blades' arrangement is shown in FIG. 27.

FIG. 32 shows the positioning of the striking members and blades of the device in FIG. 26 to FIG. 28 for deriving the W-plasty patterns in FIG. 27. Each striker head is joined (e.g., welded) to a blade. The blade width is 0.5 mm. The slit width for each blade in the jig is 1.3 mm so that each slit is a “sliding fit” for each blade to move through. The spring energy formula: 1/ 2.k. x2. Current working spring specifications: k=6218 N/m, x=0.0125 mm. Spring energy required to perforate the skin with current concept: 1/2(6218 )(0.0125)²=0.4857 J>0.4857 J (meaning the spring energy has to be 0.4857 J or more, depending on the springs used, for skin perforation).

FIG. 33 shows the position of the blades corresponding to uncompressed state (left image) and compressed state (right image) of the springs.

FIG. 34 shows an overview of the device of FIG. 26 to FIG. 33 and the components.

FIG. 35 shows the surface that aids in spring compression. FIG. 35 also shows spring chambers each housing a spring to maintain the spring concentricity (i.e., so that the spring does not shift out of place when operating between the compressed and uncompressed states.

FIG. 36 shows the device of FIG. 26 to FIG. 35 having a secondary depth stopper defined by the surface of the upper member that is in contact with the lower surface of the elevation member (i.e., elevating plate).

FIG. 37 demonstrates the spring actuation method for the device in FIG. 26 to FIG. 36. In the left image, there is a stud shown, which restrains (acts as a constraint) the movement of the keyhole plate (e.g., in a single direction). Sequential firing of the strikers and blades may be realized by staggering the positions of the holes in the keyhole plate (left bottom image). The right top and right bottom images show the locked and unlocked positions, respectively, of the strikers and blades by the holes of the keyhole plate. The force required to overcome friction (keyhole plate and contact plate are made with mirror finish appearance): Normal reaction force applied by the springs: 8*(6218)(0.0125)=621.8 N, and the push force by user is then 621.8 N * friction coefficient between metal finish plate (Taking value to be approximately 0.1) which is equal to 62.18 N.

FIG. 38 shows the safety pin for preventing misfiring of the strikers and blades.

FIG. 39 shows the mechanical leverage for the spring actuation method. The lever may be coupled to pivot at the top of the device and having an element arranged to actuate the keyhole plate when a user exerts a force at the other end (an end which is distal from the pivot) of the device.

FIG. 40 shows the safety features of devices of the present disclosure. Particularly, for the device in FIG. 26 to FIG. 40, all three safety features are included.

FIG. 41 shows different geometric arrangements of the blades for the W-plasty configuration for a 2 cm scar (compared to FIG. 11, reproduced in top image). The angulation between the “V” decreases from 90 degrees to 70 degrees, The angulation at the end of the W-plasty decreases from 120 degrees to 115 degrees.

FIG. 42 shows the change in blades used (compare blade in leftmost image to the blade in bottom rightmost image in FIG. 17, reproduced in leftmost image in FIG. 42). The blade shown in FIG. 42 confers an improved cut clinically, increase the amount space in the device (i.e., the blades are “space-saving” and reduce the number of components, as multiple blades may correspond to one striker. In this non-limiting example, for the device shown in FIG. 26 to FIG. 42, instead of 14 strikers and 14 springs, only 8 strikers and 8 springs are configured.

FIG. 43 shows the contact plate 5006 (e.g., a metal contact plate) and the biasing member 5008b (e.g., keyhole plate), which have a mirror-polish finishing. Having the plates polished helps reduce the amount of static friction, which increase ease of use to activate and actuate the springs. For example, the static friction coefficient between unpolished and polished steel plates is 0.4 to 0.5 and 0.01 to 0.05, respectively.

FIG. 44 is a table summarizing further configurations/features applicable to the device of FIG. 26 to 43.

FIG. 45 shows a device based on that of FIG. 26 to FIG. 43, except that instead of eight strikers, two strikers are used for rendering the same plasty configuration (cut pattern).

FIG. 46 shows one set of blades is welded to one striking member and another set of blades is welded to another striking member, such that only two strikers are needed. The previous configuration in the device of FIGS. 26 to 43 is shown in the right images.

FIG. 47A shows a perspective view of the device of FIG. 45 and FIG. 46. The reference FIG. 47B is a table indicating the component that corresponds to the reference numeral in FIG. 47A.

FIG. 48 shows the details of the device in zone 1 of FIG. 45.

FIG. 49A shows the details of the device in zone 2 of FIG. 45. Left image shows the bushing and right image shows the spring's positioning when compressed and uncompressed. The bushing helps to constrain and guide the strikers to move strictly in the vertical (up and down motion) axis. The bushing system can be made of PTFE (polytetrafluoroethylene), which helps to reduce friction as the strikers slide down after the activation of springs. The spring energy: 1/2 k(x)2=0.5409 J (two blades). One blade, may require 0.27 J. The striker has 8 blades attached, therefore the spring energy required: 0.27×8=2.16 J. Spring used/to be used: Outer diameter: 9.6 mm, inner diameter: 6.4 mm, spring constant: 1521 kg/m, given 14 mm compression, spring energy: 1/2 (1521)(9.81)(0.833)×(0.014)\hat{ }2=2.43 J.

FIG. 49B shows the details of the device in zone 2 of FIG. 45, particularly the spring, the strikers and welded blades.

FIG. 50 shows the staggered configuration in the blades. The blades are arranged side by side as shown in the other prototype (shown in FIG. 11 and FIG. 13). To sharpen the blade tip further, the blades are configured to stagger as shown in the top image (by 1 mm), which improves the cutting efficacy even more.

FIG. 51 shows the blade configuration and a placement jig (i.e., bottom member). In the device of FIG. 44 to current FIG. 51, a modified crescent blade is used. The top angle is further reduced from 37° to 25°.

### DETAILED DESCRIPTION

The following detailed description refers to the accompanying drawings that show, by way of illustration, specific details and embodiments in which the present disclosure may be practised.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

The present disclosure relates to a device for perforating a skin (e.g., for biopsy and scar revision). The device is versatile in that it can include configurations of various components, rendering various versions of the device. Advantageously, the present device reduces the time and skill level required for such operations.

The present device is able to produce a cut pattern (e.g., W-plasty geometric cut) in a significantly shorter time (e.g., less than 5 seconds), more accurately and conveniently than traditional devices and methods. The present device may be referred to as a pre-loaded (with an actuation mechanism) cutting device with the blades aligned in the right orientation and the right force to penetrate the dermis to the correct depth to produce a clean plasty-cut (e.g., W-plasty cut). There is also a mechanism to prevent the blades from cutting too deep.

The present device, for hygiene considerations, can be a single-use device to remove tissue with ease, precisely, safely and within the time frame of 10 minutes or less. Moreover, the area of cut is quite big in comparison to biopsy punches and the accuracy of the cuts need to be of similar consistency as compared to what an experienced surgeon is capable of producing when using traditional devices and methods.

The present device is able to produce a plasty cut (e.g., W-plasty cut) with minimal cuts, saving time, hassle and prevent excessive blood from oozing out.

The components of the present device are advantageously configured to deal with scar lengths of, for example, 3.5 cm to 5 cm, which covers about 90% of the market requirements and demands. The components of the present device are configured, and may be made of material, such that there is good visibility of the cutting site and scar to be revised.

The present device is able to minimize uneven cuts.

The present device can be operated by a single user, and even by a single hand.

The present device is compatible with wavy blades, as long as the cuts formed are symmetrical to each other.

The present device is able to produce cut depth of at least 4 mm deep and not beyond 8 mm so as to render a sufficient cut without damaging the underlying layers beneath the skin.

The present device is compatible with use of lubrication for the internal mechanism for ease of firing.

Details of various embodiments of the present device, and advantages associated with the various embodiments are now described below. Where the embodiments and advantages are illustrated in the drawings and/or described above, they shall not be iterated for brevity.

The present disclosure describes a device operable to perforate a skin. The device can be operable for skin biopsy and scar revision. The term “perforate” refers to creating of holes or punctures. The action of perforating may involve making a series of small, closely spaced holes or slits. As the device can be used to perforate a skin, the device may be termed a “skin puncher cutter”.

In general, the device is operable to create a cut pattern that is not linear, i.e., the cut is not a linear line. Said differently, the device can render any zig-zag line cut (i.e., zig-zag cut pattern). The device renders such cut pattern by short segmental cuts (i.e., segmented perforations derived from perforations of more than one blades, wherein the more than one blades are arranged to render such segmented perforations). For example, these cut segments may be created by the blades of the device (which are smaller compared to traditional device) that may be operated sequentially in a short time, less than 5 seconds. The period between each segment (i.e., interval between each perforation) may be pre-defined by the mechanism and components of the device (e.g., an actuation mechanism, a biasing member, etc.). The blades that render the cut segments may utilize a “stored” energy, such as that from one or more compressed springs, pressured gas, or an electric or magnetic form of stored energy that can be converted to kinetic energy (i.e., to drive the blades to perforate the skin). The actuation mechanism for utilizing any of aforesaid “stored” energy can be from any energy sources such as a mechanical spring, an electrical source or a magnetic source. The device may be operable for scar revision, biopsy, or any other applications requiring to cut the skin in a non-linear fashion.

In various embodiments, the device may comprise a housing comprising an upper member and a bottom member, wherein the upper member may be detachably engaged to the bottom member.

In various embodiments, the device may comprise an actuation mechanism housed proximal to the upper member.

In various embodiments, the device may comprise a biasing member operably coupled to the actuation mechanism. The actuation mechanism comprises an actuating member (e.g., a spring). Other than a spring, the actuation mechanism may involve use of other modes of energy to render the perforations by the blades. Such actuation mechanisms may involve the use of pressured gas, an electric or a magnetic source capable of rendering the same effect (other forms of energy converted to kinetic energy, i.e., movement of the blades) as the spring.

In various embodiments, the device may comprise one or more striking members, and more than one blades, each of the more than one blades operably coupled to a corresponding striking member of the one or more striking members. Said differently, each blade can be coupled to a respective striking member (without being limited to one striking member per blade). Each striking member need not be restricted to be operably coupled to one blade, rather, each blade can be operably coupled to a striker that is operably coupled to another blade (i.e., one striking member two blades). Further, in various embodiments described below, each striking member may be coupled to an actuating member (e.g., a spring). With this, it is understandable that (i) each blade may be operably coupled to one spring (e.g., one spring per blade), and (ii) multiple blades may be operably coupled to one spring (e.g., one spring two blades). In various embodiments, the device may comprise more than one striking members, and more than one blades, each of the more than one blades operably coupled to a corresponding striking member of the more than one striking members. In various embodiments, the more than one blades may be arranged to render perforation(s) having a pattern (also termed herein as cut pattern) that is non-linear. The pattern may be segmented (i.e., has various segments forming the pattern). Said differently, the more than one blades may be arranged to render segmented perforations.

In various embodiments, the biasing member may be operable to have the actuation mechanism activate an actuating member to drive each of the one or more striking members in a manner which renders each of the more than one blades to penetrate a skin. In various embodiments, the biasing member may be operable to have the actuation mechanism activate an actuating member to drive each of the more than one striking members in a manner which renders each of the more than one blades to perforate the skin (e.g., penetrate the skin).

In various embodiments, the device may further comprise an intermediate member configured between and detachably engaged to the upper member and bottom member, an elevation member, and a top cover and a contact plate configured between the elevation member and the top cover. The elevation member may be detachably engaged to the upper member and configured between the top cover and the upper member.

In various embodiments, the biasing member may be configured between the elevation member and the top cover. The biasing member may comprise moveable plates. Each of the moveable plates may comprise a grip element for a user to push one or more of the moveable plates independently (e.g., sequentially in any sequence desired, such as from left to right, right to left, or starting with one of the moveable plates in between) or simultaneously. Understandably, such operation of the one or more moveable plates translates to independent (e.g., sequential) or simultaneous perforations from the more than one blades. For example, a user may operate one grip element to render one of the moveable plates to have one or more blades perforate the skin. The user may then operate another grip element (e.g. adjacent or non-adjacent to the grip element that has been operated) to render another of the moveable plates to have one or more blades perforate the skin. The user may continue in such sequence until all grip elements and hence all moveable plates (and the blades) are operated on. In another example, the user may choose any grip element to start with, and independently choose any other grip element to operate on. In another example, the user may operate all grip elements to have all moveable plates simultaneously operated, which in turn renders all blades to perforate the skin simultaneously. Advantageously, the present device is operably versatile as to whether perforations are independently (e.g., sequentially) or simultaneously rendered. Advantageously, having grip elements that can be independently (e.g., sequentially) operable, the present device is able to render perforation from one blade independent of another, which mitigates a skin surface with uneven contours. This is because for an uneven skin surface, with a traditional device, if one blade is rendered to contact a higher contour on the skin while another blade is to perforate a lower contour on the skin, the blade perforating the higher contour may generate an impact that affects perforation of the blade perforating the lower contour, e.g., the impact may de-stabilize the device or disturb the skin surface, such that under-or over-perforation at the lower contour may occur. Hence, the present device is able to circumvent such undesirable perforations. Also, the driving force of each blade into the skin is independent of another (not held back or affected by another blade when operated independently or sequentially). Advantgeously, for a simultaneous operation, multiple perforations can be rendered all at one time, reducing discomfort and/or pain. Each of the moveable plates may comprise one or more keyholes and each of the moveable plates are position-able in a first state or in a second state, wherein in the first state the moveable plates may be positioned to withhold the more than one blades from penetrating the skin, and wherein in the second state the moveable plates may be positioned to have the one or more keyholes align with each corresponding striking member of the one or more (e.g., more than one) striking members to be driven through by the actuating member.

In various embodiments, each of the one or more keyholes may be shaped to (i) withhold the one or more (e.g., more than one) striking members from being driven through by the actuating member in the first state and (ii) have each corresponding striking member of the one or more (e.g., more than one) striking members passable through when driven by actuating member in the second state.

In various embodiments, the actuating member may comprise a spring. The spring may be wrapped around each of the one or more (e.g., more than one) striking members as described in various embodiments mentioned above and below. The spring may be held in a compressed state between the biasing member and a top portion of the upper member and the spring may be released from the compressed state when the biasing member is pushed (or pulled) to release the spring from the compressed state. Other than a spring, the actuating member may involve use of other modes of stored energy to render the perforations by the blades, for example, pressured gas, an electric or a magnetic source capable of rendering the same effect (other forms of stored energy converted to kinetic energy, i.e., movement of the blades) as the spring.

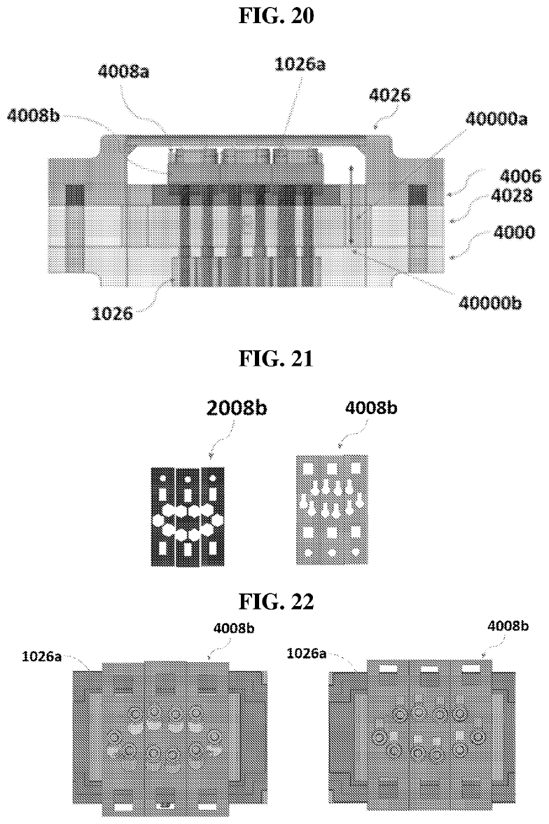

In various embodiments, the one or more (e.g., more than one) striking members may be stopped at a surface of the top portion (of the upper member) and/or at a surface of a bottom portion of the upper member. To aid the understanding of this feature, this is illustrated and described by way of a non-limiting example in the brief description section for FIG. 20.

In various embodiments, the bottom member may comprise slits configured in line with an arrangement of the more than one blades for forming a cut pattern (e.g., a W-plasty pattern) on the skin. In various embodiments, the bottom member may comprise slits configured to have the more than one blades form a W-plasty pattern on the skin.

In various embodiments, the device may further comprise an intermediate member configured between and detachably engaged to the upper member and bottom member.

In various embodiments, the biasing member may be configured between the upper member and the intermediate member. The biasing member may comprise moveable plates. Each of the moveable plates may comprise a grip element for a user to push or pull one or more of the moveable plates independently (e.g., sequentially in any sequence desired, such as from left to right, right to left, or starting with one of the moveable plates in between) or simultaneously. Understandably, such operation of the one or more moveable plates translates to independent (e.g., sequential) or simultaneous perforations from the more than one blades. For example, a user may operate one grip element to render one of the moveable plates to have one or more blades perforate the skin. The user may then operate another grip element (e.g. adjacent or non-adjacent to the grip element that has been operated) to render another of the moveable plates to have one or more blades perforate the skin. The user may continue in such sequence until all grip elements and hence all moveable plates (and the blades) are operated on. In another example, the user may choose any grip element to start with, and independently choose any other grip element to operate on. In another example, the user may operate all grip elements to have all moveable plates simultaneously operated, which in turn renders all blades to perforate the skin simultaneously. Advantageously, the present device is operably versatile as to whether perforations are independently (e.g., sequentially) or simultaneously rendered. Advantageously, having grip elements that can be independently (e.g., sequentially) operable, the present device is able to render perforation from one blade independent of another, which mitigates a skin surface with uneven contours. This is because for an uneven skin surface, with a traditional device, if one blade is rendered to contact a higher contour on the skin while another blade is to perforate a lower contour on the skin, the blade perforating the higher contour may generate an impact that affects perforation of the blade perforating the lower contour, e.g., the impact may de-stabilize the device or disturb the skin surface, such that under-or over-perforation at the lower contour may occur. Hence, the present device is able to circumvent such undesirable perforations. Also, the driving force of each blade into the skin is independent of another (not held back or affected by another blade when operated independently or sequentially). Advantgeously, for a simultaneous operation, multiple perforations can be rendered all at one time, reducing discomfort and/or pain. Each of the moveable plates may comprise one or more keyholes and each of the moveable plates are position-able in a first state or in a second state, wherein in the first state the moveable plates may be positioned to withhold the more than one blades from penetrating the skin, and wherein in the second state the moveable plates may be positioned to have the one or more keyholes align with each corresponding striking member of the one or more (e.g., more than one) striking members to be driven through by the actuating member.

In various embodiments, the actuating member may be or may comprise a spring. The spring may be wrapped around each of the one or more (e.g., more than one) striking members as described in various embodiments mentioned above and below. The spring may be held in a compressed state between the biasing member and a top portion of the upper member and the spring may be released from the compressed state when the biasing member is pulled or pushed to release the spring from the compressed state. Other than a spring, the actuating member may involve use of other modes of stored energy to render the perforations by the blades, for example, pressured gas, an electric or a magnetic source capable of rendering the same effect (other forms of stored energy converted to kinetic energy, i.e., movement of the blades) as the spring.

In various embodiments, the bottom member may comprise slits configured in line with the arrangement of the more than one blades for forming a cut pattern (e.g., a W-plasty pattern) on the skin. In various embodiments, the bottom member may comprise slits configured to have the more than one blades form a W-plasty pattern on the skin.

In various embodiments, the device may further comprise a contact plate configured between the upper member and the intermediate member.

In various embodiments, the bottom member may comprise slits (e.g., two slits) configured to have the more than one blades form a cut pattern (e.g., V-shaped cut) on the skin, and/or the upper member may comprise two opposing openings shaped to hold the biasing member abut against the actuating member.

In various embodiments, the biasing member and the two opposing openings may be structurally configured to have the biasing member inserted through the upper member without the biasing member entirely passing through the two opposing openings. In various embodiments, the biasing member is removable in a manner to have the more than one blades perforate the skin independently (e.g., sequentially) or simultaneously. This can be done by removing the biasing member rapidly such that the blades are driven simultaneously, or removing the biasing member to have one of the blades driven followed independently (e.g., sequentially) by another blade. For example, a user may operate one biasing member to have one of the blades perforate the skin. The user may then operate another biasing member (e.g. adjacent or non-adjacent to the biasing member that has been operated) to render another blade perforate the skin. The user may continue in such sequence until all biasing members and hence all blades are operated on. In another example, the user may choose any biasing member to start with, and independently choose any other biasing member to operate on. In another example, the user may operate all baising members to have all blades simultaneously perforate the skin. Advantageously, the present device is operably versatile as to whether perforations are independently (e.g., sequentially) or simultaneously rendered. Advantageously, having biasing members that can be independently (e.g., sequentially) operable, the present device is able to render perforation from one blade independent of another, which mitigates a skin surface with uneven contours. This is because for an uneven skin surface, with a traditional device, if one blade is rendered to contact a higher contour on the skin while another blade is to perforate a lower contour on the skin, the blade perforating the higher contour may generate an impact that affects perforation of the blade perforating the lower contour, e.g., the impact may de-stabilize the device or disturb the skin surface, such that under-or over-perforation at the lower contour may occur. Hence, the present device is able to circumvent such undesirable perforations. Also, the driving force of each blade into the skin is independent of another (not held back or affected by another blade when operated independently or sequentially). Advantgeously, for a simultaneous operation, multiple perforations can be rendered all at one time, reducing discomfort and/or pain.

In various embodiments, the upper member may comprise a window. The window may be configured at a portion of the upper member which is detachably engaged to the bottom member. For a better understanding, the window is illustrated and described by way of a non-limiting example in the brief description section for FIG. 2.

In various embodiments, the actuating member may comprise a spring. The spring may be wrapped around each of the more than one striking members. That is to say, each of the one or more (e.g., more than one) striking members may be wrapped around with a spring. The spring may be held in a compressed state between the biasing member and a top portion of the upper member and the spring is released from the compressed state when the biasing member is removed from the upper member. Other than a spring, the actuating member may involve use of other modes of stored energy to render the perforations by the blades, for example, pressured gas, an electric or a magnetic source capable of rendering the same effect (other forms of stored energy converted to kinetic energy, i.e., movement of the blades) as the spring.

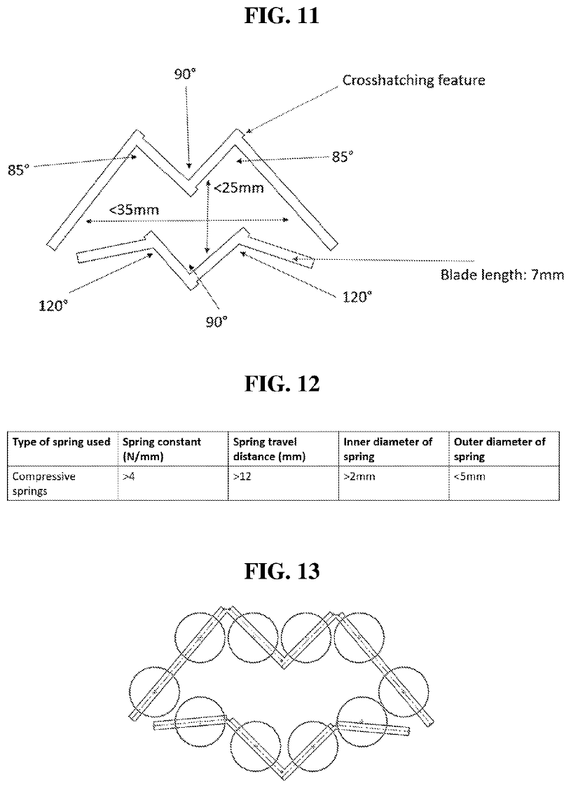

In various embodiments, the one or more (e.g., more than one) striking members, the more than one blades, and the actuating member, may be arranged for forming the cut pattern on the skin. In various embodiments, the cut pattern may be non-linear, i.e., not a linear line. In various embodiments, the cut pattern may comprise cut segments that define the cut pattern. The cut segments are perforations rendered by the more than blades, i.e. each cut segment per blade. In various embodiments, the cut pattern may comprise a length of 2 cm to 4 cm (e.g., 2 cm to 3 cm, 2.5 cm to 3 cm, 2 cm to 3.5 cm, 3 cm to 4 cm, 3.5 cm to 4 cm), and/or has a W-shaped cut pattern, a V-shaped cut pattern, a S-shaped cut pattern, or any zig-zag shaped cut pattern. The measurement for the length of the cut pattern is shown in FIG. 11.

In various embodiments, the W-shaped cut pattern may comprise an upper pattern and a lower pattern, wherein the upper pattern and the lower pattern each has two crosshatches which may be identical to render the W-shaped cut pattern symmetrical. That is to say, the upper pattern may have two crosshatches that are identical and the lower pattern may have two crosshatches that are identical. Each of the two crosshatches of the upper pattern and each of the two crosshatches of the lower pattern may be defined by an angle, wherein the angle defining each of the two crosshatches of the upper pattern is smaller than the angle defining each of the two crosshatches of the lower pattern (see FIG. 11). In various embodiments, the angle defining each of the crosshatches of the lower pattern may be 100° to 120°, 100° to 110°, 110° to 120°, etc. (see FIG. 10).

In various embodiments, each of the more than one blades may be (i) positioned correspondingly to each of the one or more (e.g., more than one) striking members or (ii) joined to each of the one or more (e.g., more than one) striking members.

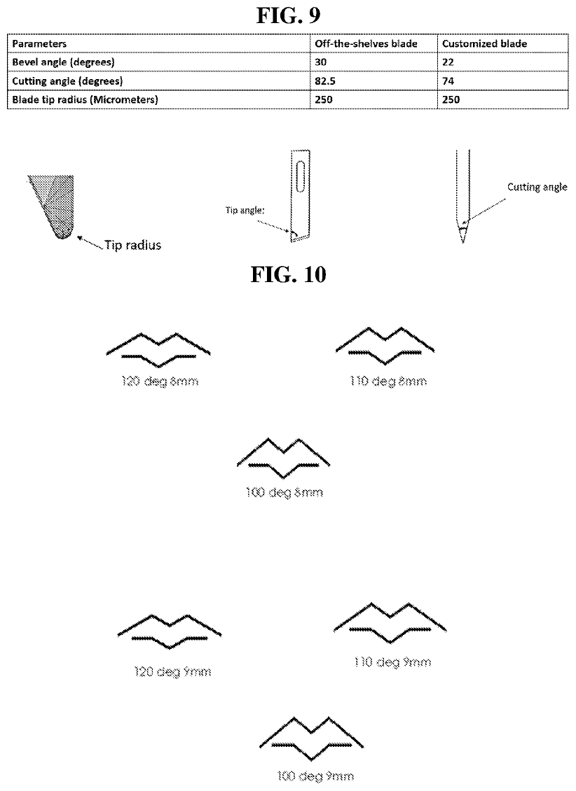

In various embodiments, each of the more than one blades may comprise a main body and an edge. The edge may be a sharp edge. The edge may extend from the main body by 2 mm to 5 mm (e.g., 2 mm to 3 mm, 2 mm to 4 mm, 3 mm to 4 mm, 3 mm to 5 mm, 4 mm to 5 mm) and has a length of 7 mm to 9 mm (e.g., 7 mm to 8 mm, 8 mm to 9 mm). The edge may have a tip configured distal from the main body. The tip (see FIG. 9) may have a bevel angle of 20° to 25° (e.g., 22°), a cutting angle of 70° to 80° (e.g., 74°), and/or a tip radius of 200 μm to 300 μm (e.g., 250 μm).

In various embodiments, the spring may render a penetration depth of 4 mm to not more than 8 mm (e.g., 5 mm to 8 mm, 6 mm to 8 mm, 7 mm to 8 mm) in the skin. In various embodiments, the actuating member may comprise a spring, the spring may comprise a spring constant of 4 N/mm or more (e.g., 5 N/mm or more, 10 N/mm or more), and/or an inner diameter of 2 mm or more (e.g., 2.5 mm or more, 3 mm or more), and/or an outer diameter of 5 mm or less (e.g., 4.5 mm or less, 4 mm or less), and/or a spring travel distance of 12 mm or more (13 mm or more, 14 mm or more).

To provide a better understanding of the device of the present disclosure and its operation, a detailed description with reference to the drawings is set out as follows. In instances where the embodiment(s) is understandable from any of the above description, they shall not be iterated for brevity.

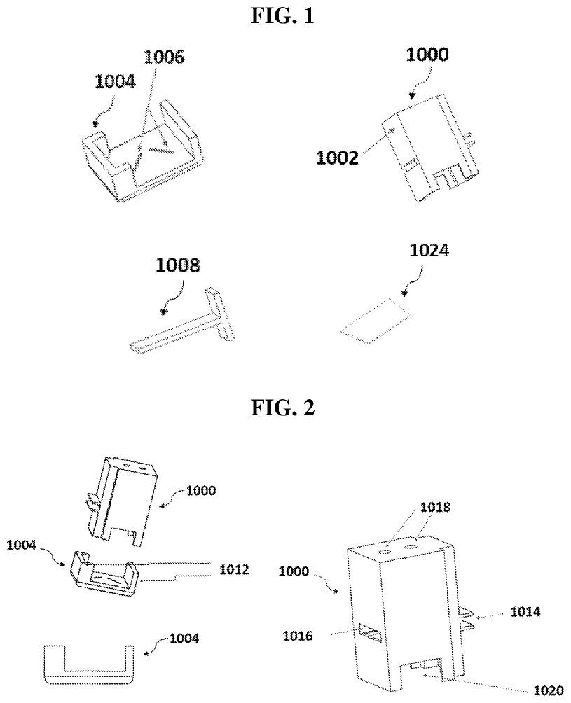

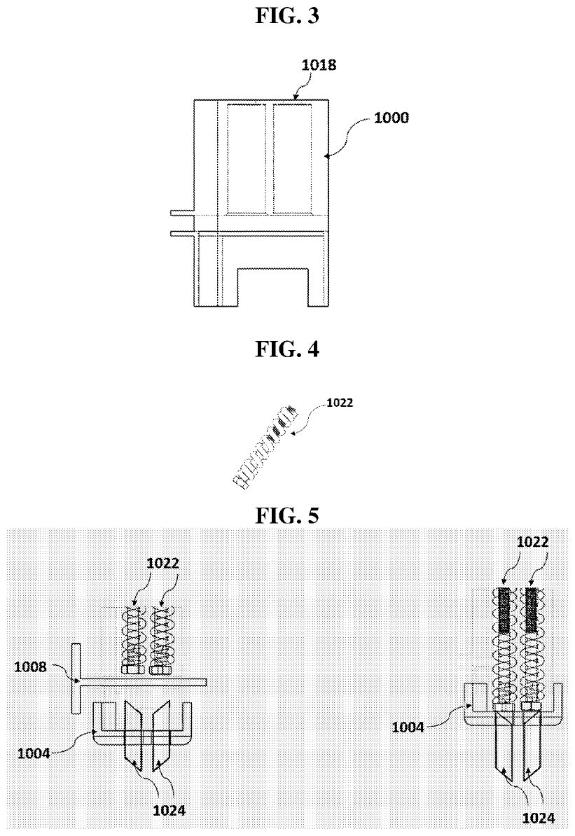

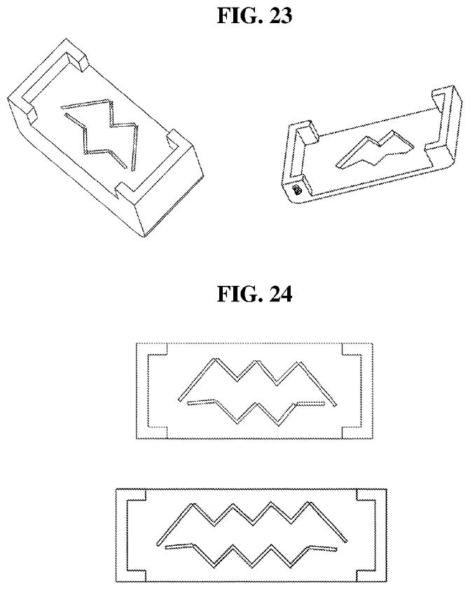

Throughout FIG. 1 to FIG. 24, it can be understood that in various embodiments, the device 10, 20, 40 may comprise a housing comprising an upper member 1000, 2000, 4000, and a bottom member 1004, 2004, 4004, wherein the upper member 1000, 2000, 4000 may be detachably engaged to the bottom member 1004, 2004, 4004. In various embodiments, the device 10, 20, 40 may comprise an actuation mechanism 1002 housed proximal to the upper member 1000, 2000, 4000. In various embodiments, the device 10, 20, 40 may comprise a biasing member 1008, 2008b, 4008b operably coupled to the actuation mechanism 1002. In various embodiments, the device 10, 20, 40 may comprise one or more striking members 1022a, 1026, and more than one blades 1024, each of the more than one blades 1024 operably coupled to a corresponding striking member of the one or more striking members 1022a, 1026. In various embodiments, the biasing member 1008, 2008b, 4008b may be operable to have the actuation mechanism 1002 activate an actuating member 1022 to drive each of the one or more striking members 1022a, 1026 in a manner which renders each of the more than one blades 1024 to perforate (e.g., penetrate) a skin 10000.

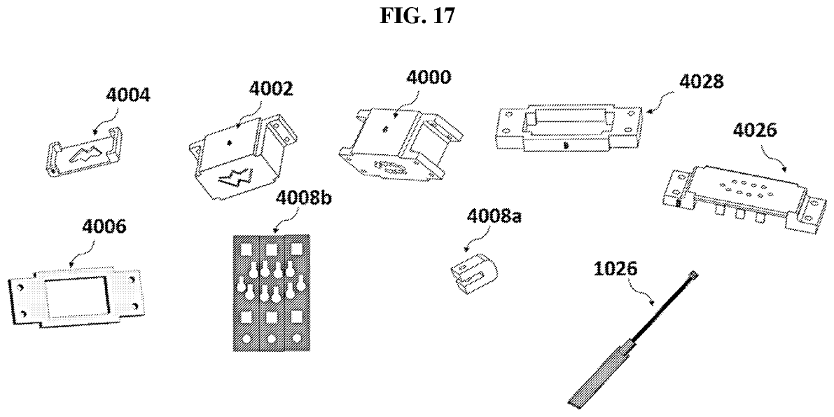

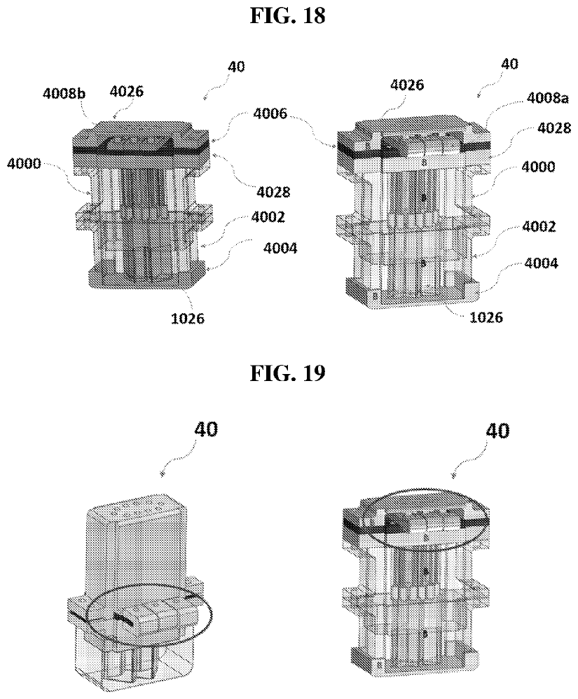

FIG. 17 to FIG. 23 demonstrate for certain non-limiting embodiments of the device. The embodiments demonstrated in these figures are non-limiting and can be applied to various embodiments of the present disclosure. The device 40 may further comprise an intermediate member 4002 configured between and detachably engaged to the upper member 4000 and bottom member 4004, an elevation member 4028, and a top cover 4026 and a contact plate 4006 configured between the elevation member 4028 and the top cover 4026. The elevation member 4028 may be detachably engaged to the upper member 4000 and configured between the top cover 4026 and the upper member 4000. FIG. 18 illustrates the device 40 assembled from the components shown in FIG. 17. It can be seen from FIG. 18 that the device includes the biasing member 4008b, which may be configured between the elevation member 4028 and the top cover 4026. The biasing member 4008b may comprise moveable plates. Each of the moveable plates may comprise a grip element 4008a for a user to push one or more of the moveable plates independently (e.g., sequentially in any sequence desired, such as from left to right, right to left, or starting with one of the moveable plates in between) or simultaneously. Understandably, such operation of the one or more moveable plates translates to independent (e.g., sequential) or simultaneous perforations from the more than one blades. Examples and advantages of such configurations and operations are already discussed above and shall not be iterated for brevity. From FIG. 17 and especially FIG. 21 to FIG. 22, it can be seen that each of the moveable plates may comprise one or more keyholes and each of the moveable plates are position-able in a first state or in a second state, wherein in the first state the moveable plates may be positioned to withhold the more than one blades (joined to the striking member 1026) from penetrating the skin (see FIG. 18 to FIG. 19 and FIG. 22 left image), and wherein in the second state the moveable plates may be positioned to have the one or more keyholes align with each corresponding striking member of the one or more (e.g., more than one) striking members 1026 to be driven through by the actuating member (see FIG. 22 right image).

From FIG. 17 to FIG. 22, it can be readily envisaged that each of the one or more keyholes may be shaped to (i) withhold the one or more (e.g., more than one) striking members 1026 from being driven through by the actuating member in the first state and (ii) have each corresponding striking member of the one or more (e.g., more than one) striking members 1026 passable through when driven by actuating member in the second state.

In the embodiments illustrated by FIG. 17 to FIG. 23, the actuating member may be understood to comprise a spring (not shown). The spring may be wrapped around each of the one or more (e.g., more than one) striking members as described in various embodiments mentioned above and below. The spring may be held in a compressed state between the biasing member 4008b and a top portion of the upper member 4000 and the spring may be released from the compressed state when the biasing member 4008b is pushed (or pulled) to release the spring from the compressed state. Other than a spring, the actuating member may involve use of other modes of stored energy to render the perforations by the blades, for example, pressured gas, an electric or a magnetic source capable of rendering the same effect (other forms of stored energy converted to kinetic energy, i.e., movement of the blades) as the spring.

From FIG. 18 and FIG. 20, it can be understood that the one or more (e.g., more than one) striking members 1026 may be stopped at a surface of the top portion (of the upper member 4000) and/or at a surface of a bottom portion of the upper member 4000. As this is already illustrated and described by way of a non-limiting example in the brief description section for FIG. 20, this shall not be iterated for brevity.

From FIG. 17 and FIG. 23 to FIG. 24, it can be seen that the bottom member 4004 may comprise slits configured in line with an arrangement of the more than one blades (joined to striking member 1026) for forming a cut pattern (e.g., a W-plasty pattern) on the skin.

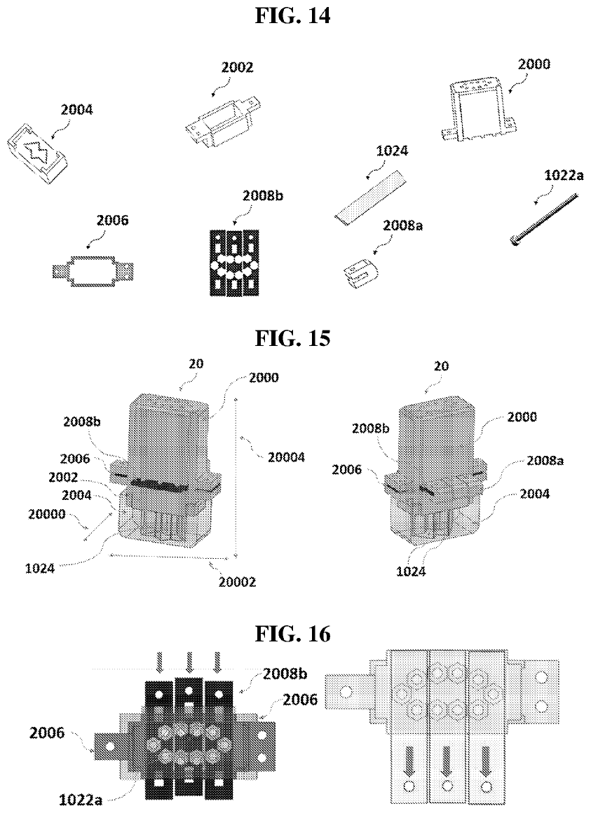

FIG. 14 to FIG. 16 demonstrate for certain non-limiting embodiments of the device. The embodiments demonstrated in these figures are non-limiting and can be applied to various embodiments of the present disclosure. The device 20 may further comprise an intermediate member 2002 configured between and detachably engaged to the upper member 2000 and bottom member 2004. The device 20 includes the biasing member 2008b, which may be configured between the upper member 2000 and the intermediate member 2002. The biasing member 2008b may comprise moveable plates. Each of the moveable plates may comprise a grip element 2008a for a user to push or pull one or more of the moveable plates independently (e.g., sequentially in any sequence desired, such as from left to right, right to left, or starting with one of the moveable plates in between) or simultaneously. Understandably, such operation of the one or more moveable plates translates to independent (e.g., sequential) or simultaneous perforations from the more than one blades 1024. Examples and advantages of such configurations and operations are already discussed above and shall not be iterated for brevity.

From FIG. 14 and FIG. 16, it can be seen that each of the moveable plates may comprise one or more keyholes and each of the moveable plates are position-able in a first state or in a second state, wherein in the first state the moveable plates may be positioned to withhold the more than one blades 1024 from penetrating the skin, and wherein in the second state the moveable plates may be positioned to have the one or more keyholes align with each corresponding striking member of the one or more (e.g., more than one) striking members 1022a to be driven through by the actuating member (not shown).

In the embodiments illustrated by FIG. 14 to FIG. 16, the actuating member may be or may comprise a spring. The spring may be wrapped around each of the one or more (e.g., more than one) striking members 1022a as described in various embodiments mentioned above and below. The spring may be held in a compressed state between the biasing member 2008b and a top portion of the upper member 2000 and the spring may be released from the compressed state when the biasing member 2008b is pulled or pushed to release the spring from the compressed state. Other than a spring, the actuating member may involve use of other modes of stored energy to render the perforations by the blades 1024, for example, pressured gas, an electric or a magnetic source capable of rendering the same effect (other forms of stored energy converted to kinetic energy, i.e., movement of the blades 1024) as the spring.

In the embodiments illustrated by FIG. 14 to FIG. 16, the bottom member 2004 may comprise slits configured in line with the arrangement of the more than one blades 1024 for forming a cut pattern (e.g., a W-plasty pattern) on the skin. In these embodiments, the device 20 may further comprise a contact plate 2006 configured between the upper member 2000 and the intermediate member 2002.

FIG. 1 to FIG. 7 demonstrate for certain non-limiting embodiments of the device. The embodiments demonstrated in these figures are non-limiting and can be applied to various embodiments of the present disclosure. In the embodiments as illustrated by FIG. 1 to FIG. 7, the bottom member 1004 may comprise slits (e.g., two slits) configured to have the more than one blades 1024 form a cut pattern (e.g., V-shaped cut) on the skin 10000, and/or the upper member 1000 may comprise two opposing openings 1014, 1016 shaped to hold the biasing member 1008 abut against the actuating member 1002 (not shown in detail).

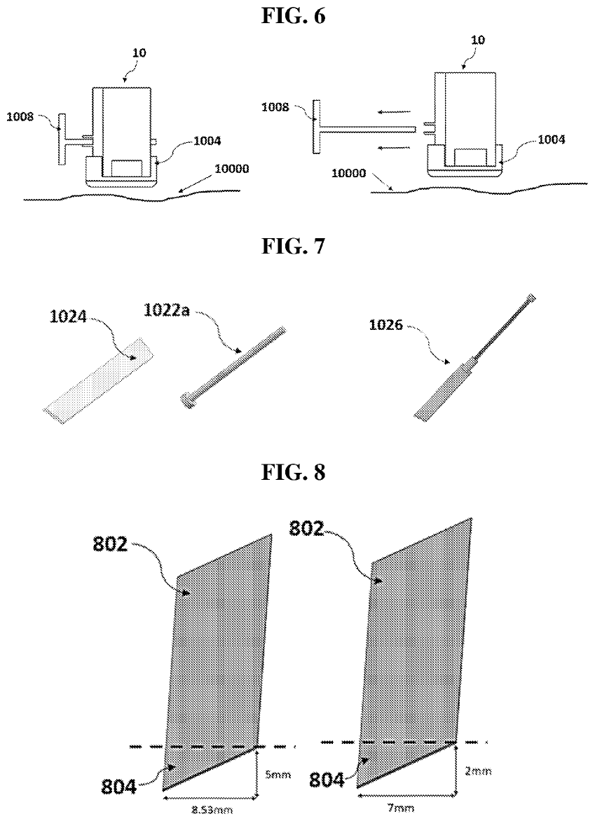

From FIG. 5 and FIG. 6, it can be seen that the biasing member 1008 and the two opposing openings 1014, 1016 may be structurally configured to have the biasing member 1008 inserted through the upper member 1000 without the biasing member 1008 entirely passing through the two opposing openings 1014, 1016.

FIG. 1 to FIG. 3, particularly FIG. 2, shows the upper member 1000 may comprise a window 1020. The window 1020 may be configured at a portion of the upper member 1000 which is detachably engaged to the bottom member 1004.

In the embodiments illustrated by FIG. 1 to FIG. 7, the actuating member may comprise a spring. The spring may be wrapped around each of the more than one striking members 1022 as shown in FIG. 4 and FIG. 5. That is to say, each of the one or more (e.g., more than one) striking members 1022 may be wrapped around with a spring. The spring may be held in a compressed state between the biasing member 1008 and a top portion of the upper member 1000 and the spring is released from the compressed state when the biasing member 1008 is removed from the upper member 1000. As can be envisaged from FIG. 1 to FIG. 3, the biasing member 1008 is removable in a manner to have the more than one blades 1024 perforate the skin independently (e.g., sequentially) or simultaneously. This can be done by removing the biasing member 1008 rapidly such that the blades 1024 are driven simultaneously, or removing the biasing member 1008 to have one of the blades 1024 driven followed independently (e.g., sequentially) by another blade 1024. Examples and advantages of such configurations and operations are already discussed above and shall not be iterated for brevity. Other than a spring, the actuating member may involve use of other modes of stored energy to render the perforations by the blades 1024, for example, pressured gas, an electric or a magnetic source capable of rendering the same effect (other forms of stored energy converted to kinetic energy, i.e., movement of the blades 1024) as the spring.

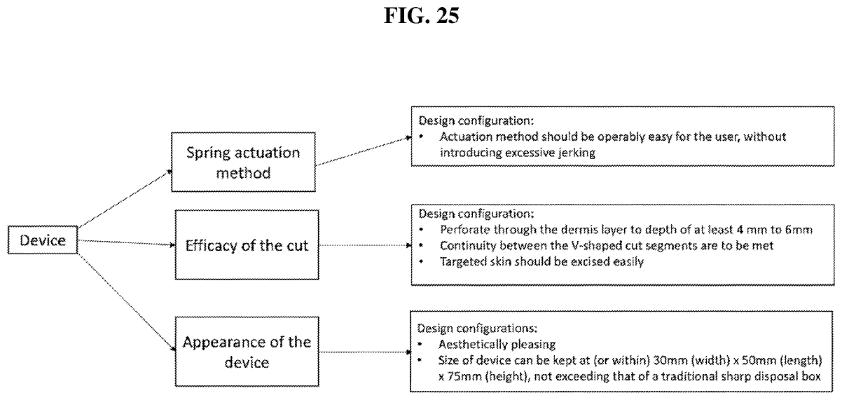

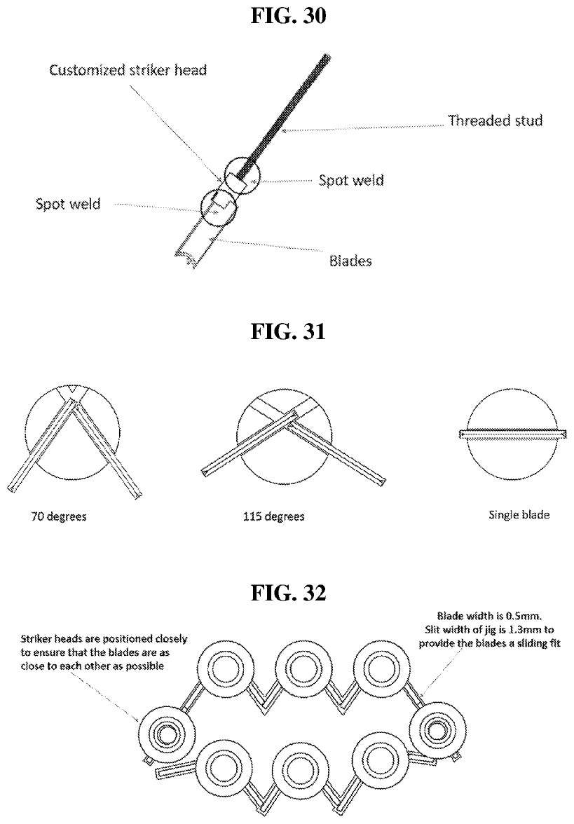

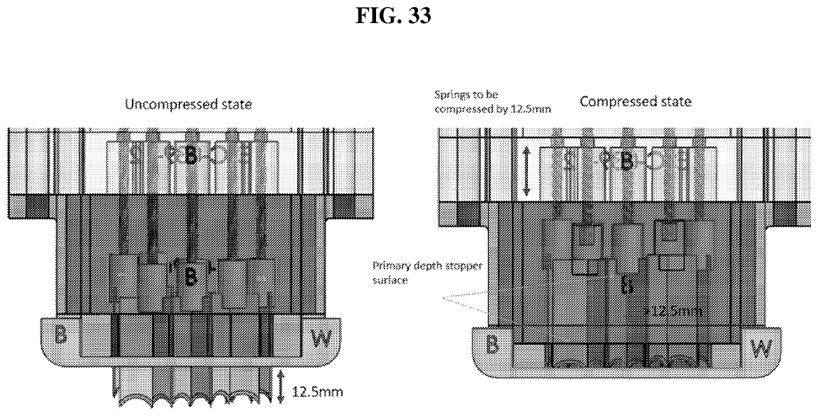

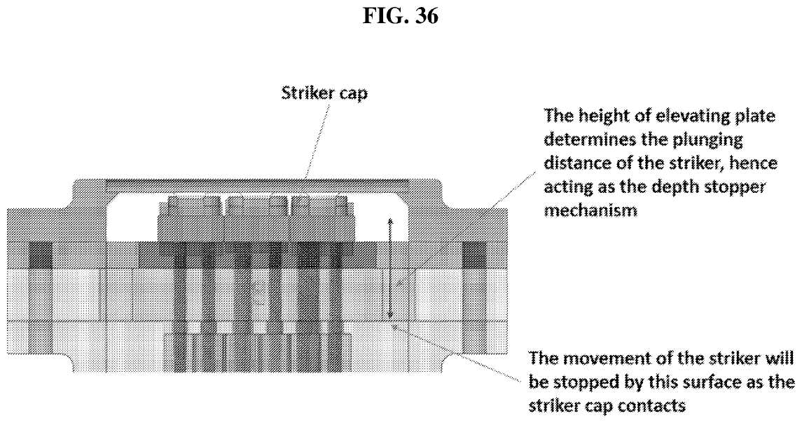

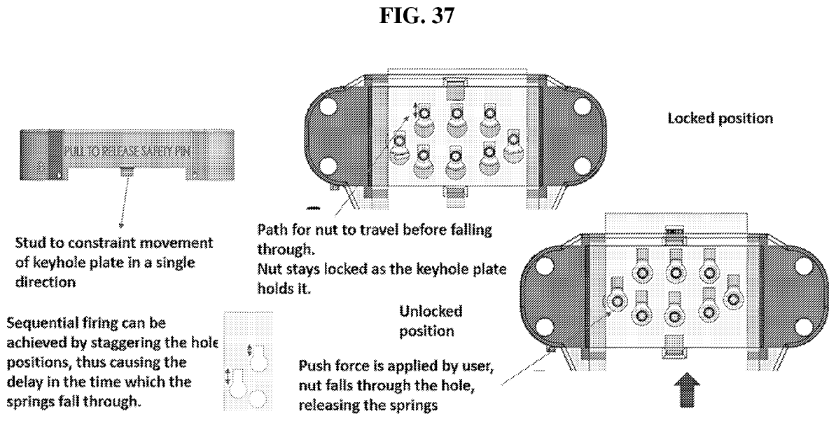

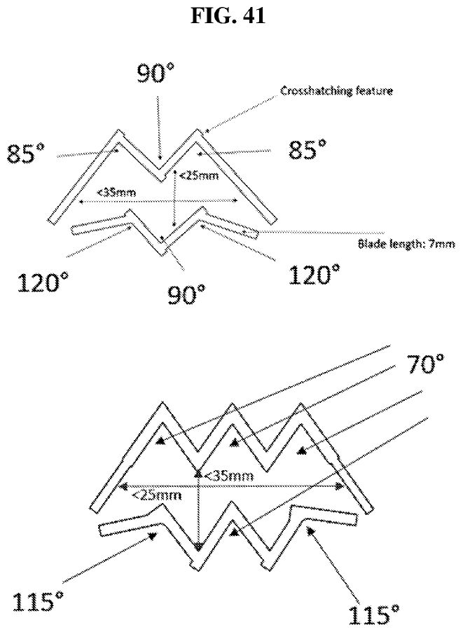

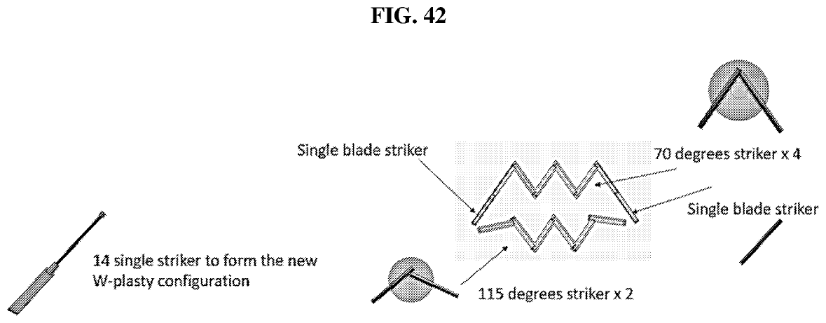

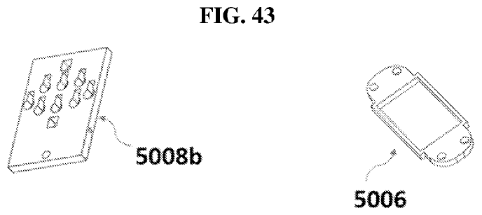

FIG. 25 to FIG. 43 relate to various embodiments of devices of the present disclosure. The devices of these embodiments may be built from the devices of one or more embodiments mentioned above. In other words, where features of devices of earlier embodiments mentioned above are present in devices of the embodiments shown by way of non-limiting examples in FIG. 25 to FIG. 43, the advantage(s) corresponding to such features are also present. For brevity, the same features or components, and the same advantages, shall not be iterated. Accordingly, FIG. 25 to FIG. 43 relate to devices that have a different geometric arrangement of blades (e.g., for forming a 2 cm scar) as described above in relation to FIG. 41. The devices of such embodiments may have a set of V-shaped joint blade strikers, as compared to single blade strikers used in one or more earlier mentioned embodiments (see FIG. 42). The devices of such embodiments include metal contact plate and keyhole plates that may have mirror-polish finishing, which reduces static friction and improve ease of activating the springs (see FIG. 43). There may be up to three level of safety features implemented in devices of such embodiments (see FIG. 40). Devices of such embodiments may also utilize an amount of spring energy for perforating the skin as compared to one or more earlier mentioned embodiments (see description for FIG. 31 and FIG. 37). Devices of such embodiments may have a single keyhole push plate instead of more than one keyhole push plates (see FIG. 37). Devices of such embodiments may have a lever (see description for FIG. 39).

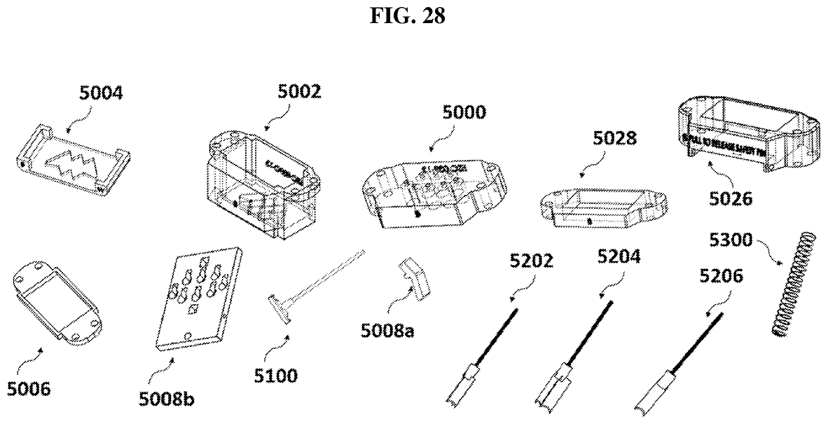

In various embodiments, the biasing member may comprise one moveable plate (see FIG. 28 and FIG. 37), instead of moveable plates, wherein the one movable plate may be coupled to a removable safety pin which is configured to restrain movement of the one moveable plate until the safety pin is removed from the device.

In various embodiments, the biasing member may comprise keyholes each positioned in alignment with a corresponding striking member so as to have all striking members pass through the keyholes simultaneously or have each of the corresponding striking member pass through the keyholes sequentially.

In various embodiments, the more than one blades may be welded to a corresponding striking member.

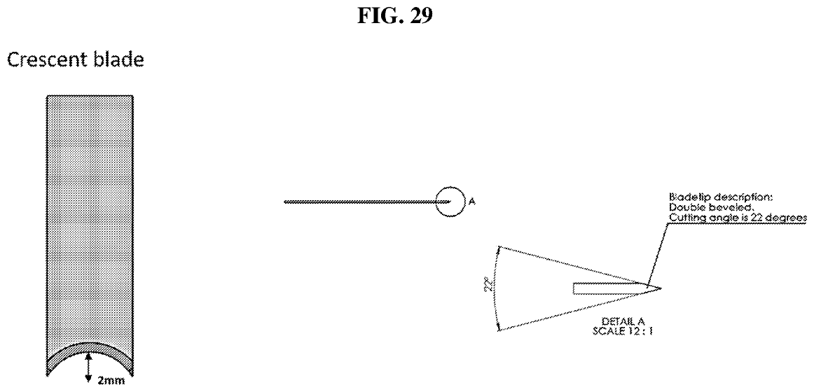

In various embodiments, each of the more than one blades may comprise a blade tip having a crescent shape defined by a concave portion and two tips, wherein the two tips may have a bevel angle of 37°, and wherein the blade tip may have a cutting angle of 22° (see FIG. 29).

In various embodiments, the one or more striking members, the more than one blades, and the actuating member, may be arranged for forming the cut pattern on the skin, wherein the cut pattern is non-linear and comprises cut segments which define the cut pattern.

In various embodiments, the cut pattern may comprise a length of 2 cm to 4 cm, and/or may have a W-shaped cut pattern.

In various embodiments, the W-shaped cut pattern may comprise an upper pattern and a lower pattern, wherein the upper pattern and the lower pattern each may have three crosshatches which may be identical to render the W-shaped cut pattern symmetrical, and wherein each of the three crosshatches of the upper pattern and each of the three crosshatches of the lower pattern may be defined by an angle, wherein the angle defining each of the three crosshatches of the upper pattern may be smaller than the angle defining two of the crosshatches of the lower pattern and may be identical to one angle defining one of the crosshatches of the lower pattern (see FIG. 41, e.g., bottom image).

In various embodiments, the angle defining each of the three crosshatches of the upper pattern may be 70° and the angle defining two of the crosshatches of the lower pattern may be 115° (see FIG. 27).

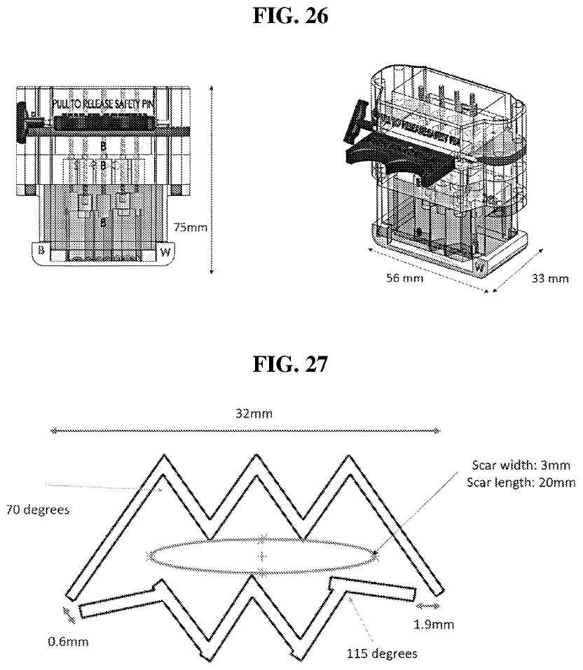

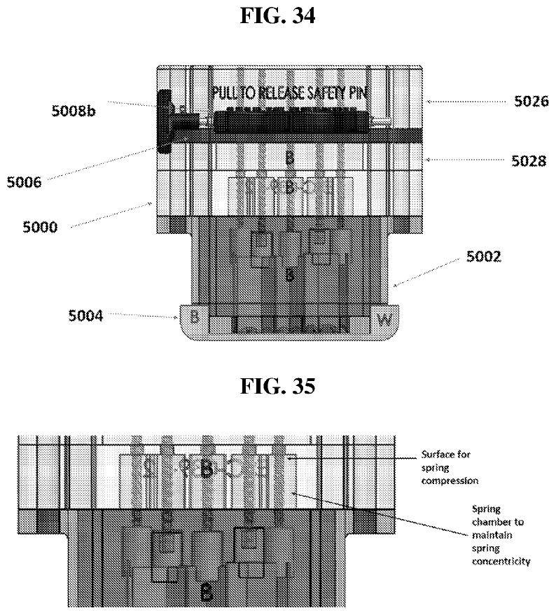

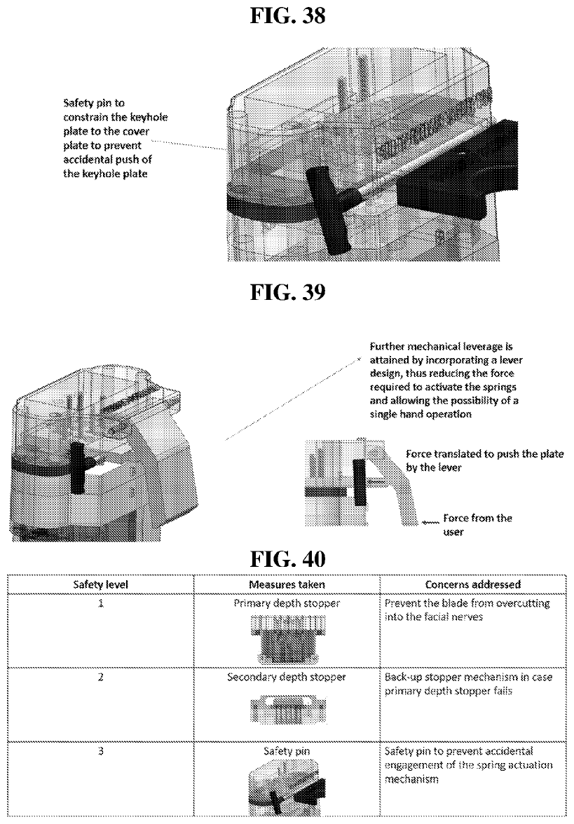

In various embodiments, the device may further comprise a lever having one end rotatably coupled to the top cover, and a portion which contacts the one moveable plate, wherein the lever may be operable to have the portion push against the one moveable plate, after the removable safety pin may be removed (see FIG. 26, FIG. 34, and FIG. 38).

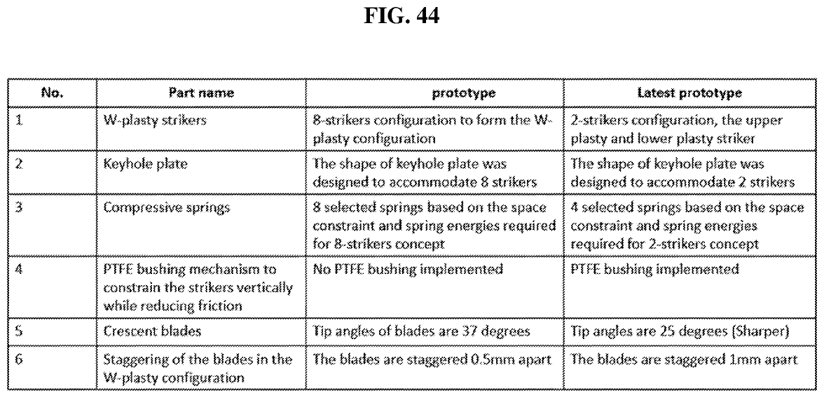

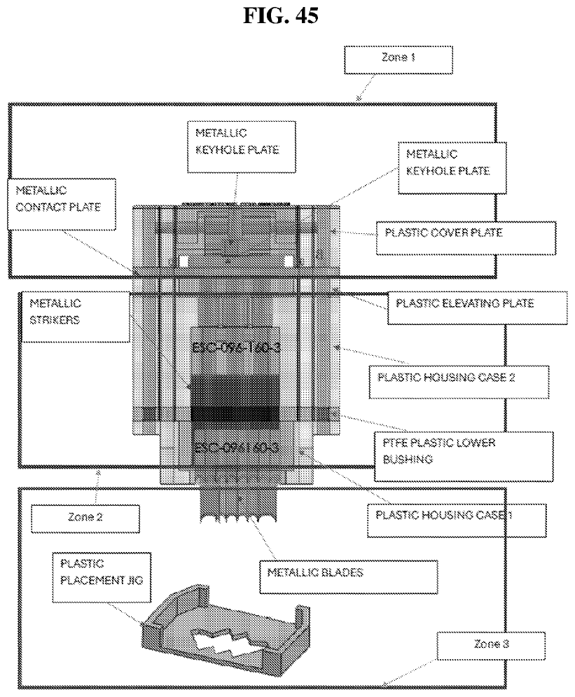

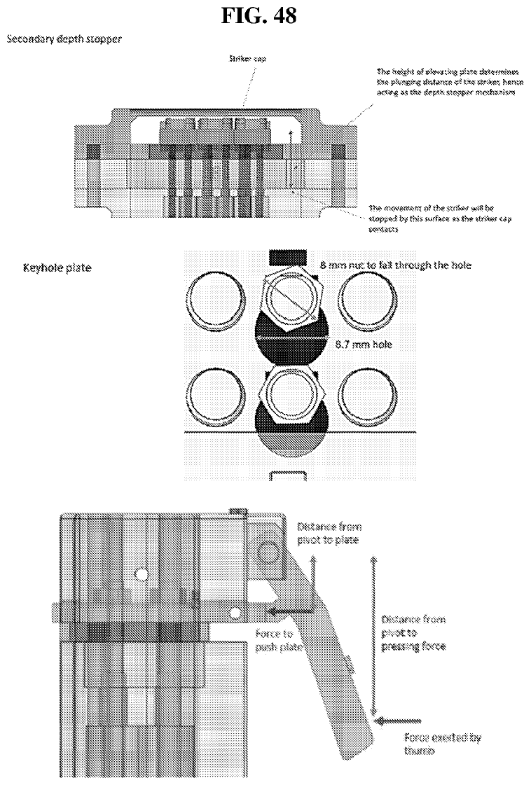

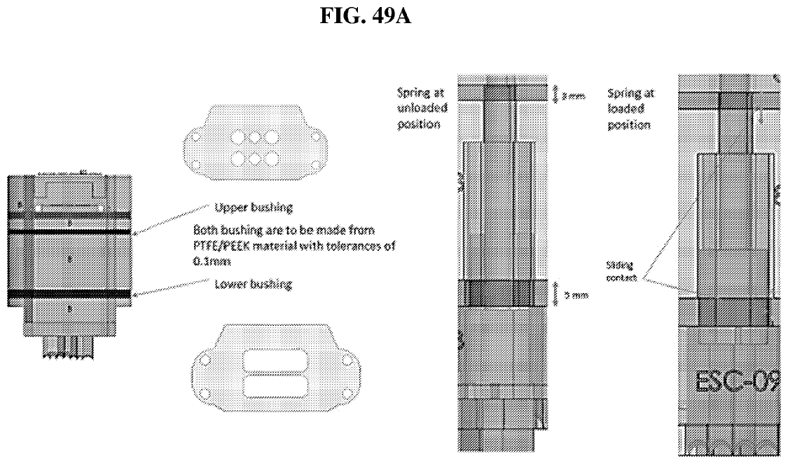

FIG. 44 to FIG. 51 relate to various embodiments of a device of the present disclosure. The devices of these embodiments may be built from the devices of one or more embodiments mentioned above. In other words, where features of devices of earlier embodiments mentioned above are present in devices of the embodiments shown by way of non-limiting examples in FIG. 44 to FIG. 51, the advantage(s) corresponding to such features are also present. For brevity, the same features or components, and the same advantages, shall not be iterated. Accordingly, a summary of certain non-limiting differences between the devices of FIG. 25 to FIG. 43 and the devices of FIG. 44 to FIG. 51 are summarized in FIG. 44.

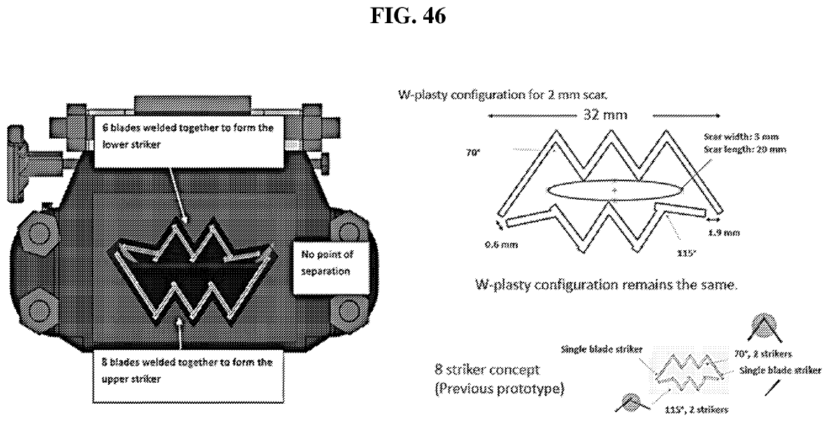

In various embodiments, a set of the more than one blades may be welded to one striking member and another set of the more than one blades may be welded to another striking member so as to have two striking members in the device (see FIG. 46).

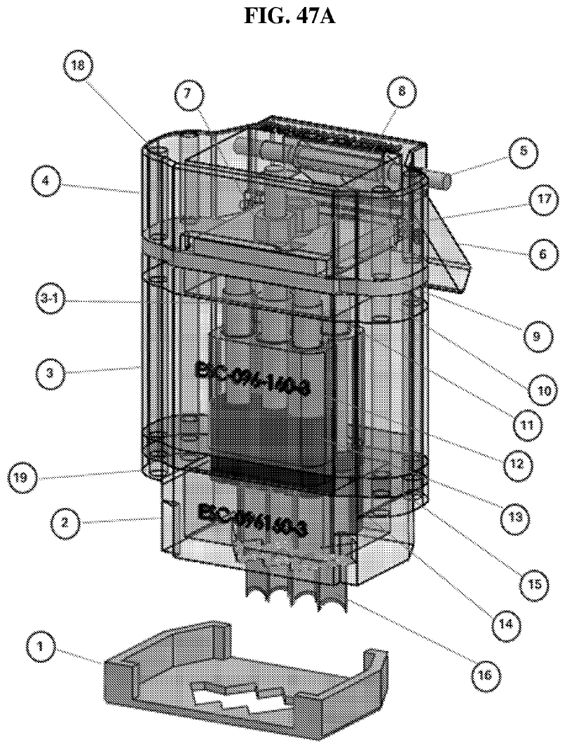

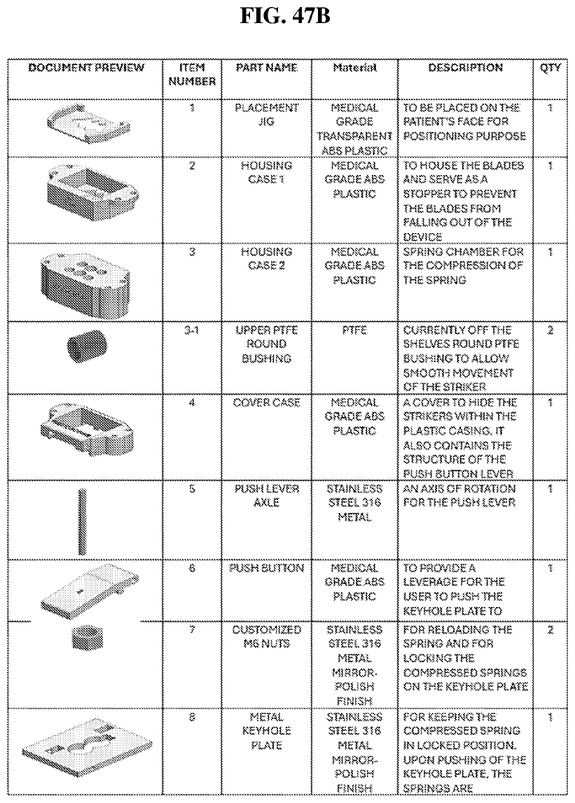

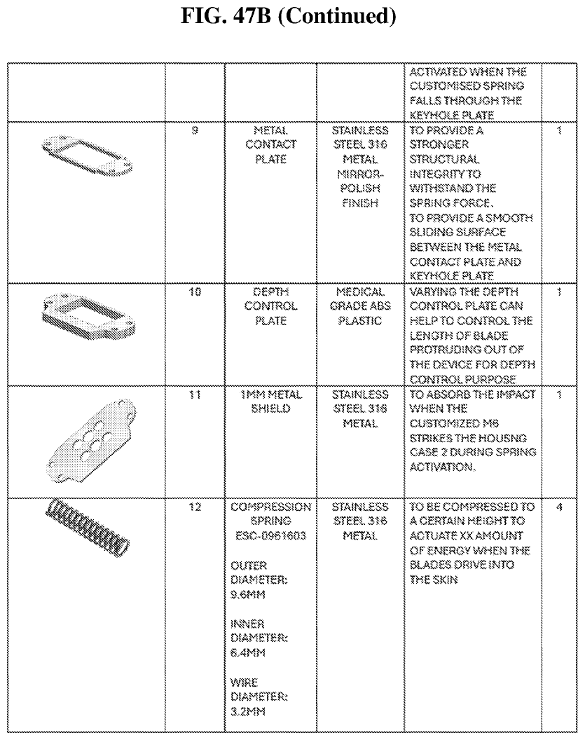

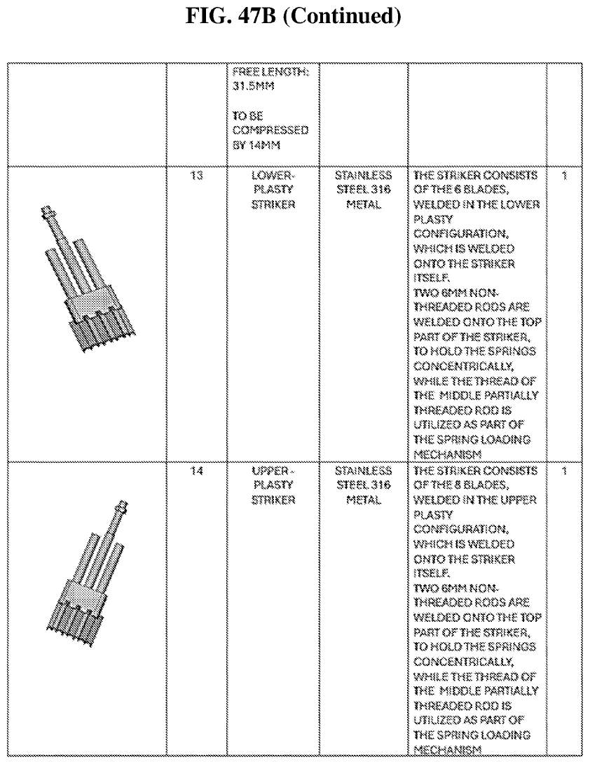

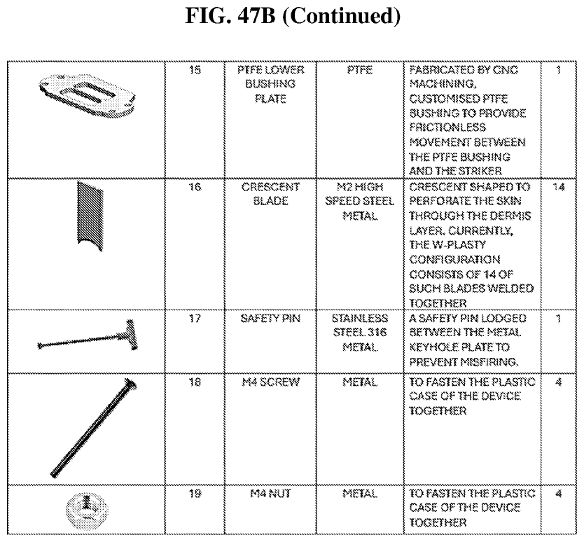

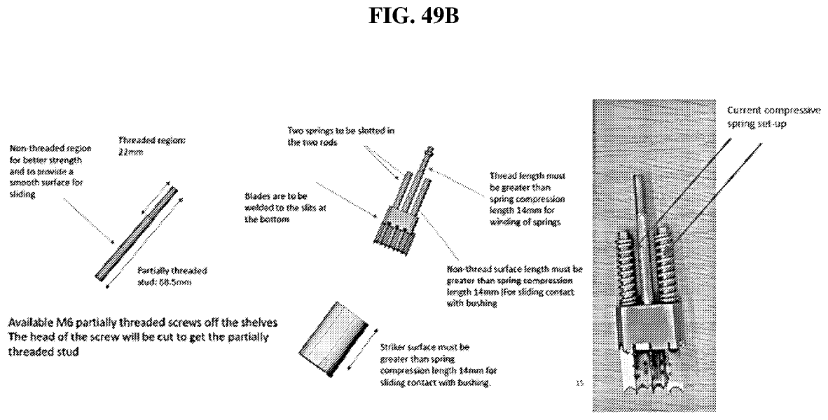

In various embodiments, wherein the striking member may comprise one extension structure longer than two other extension structures (see FIG. 47B, reference numerals 13 and 14), wherein the two other extension structures may each be coupled with one spring (see FIG. 49B).

In various embodiments, the device may further comprise a lower bushing configured between the upper member and the bottom member (see FIG. 49A), and an upper bushing configured at the upper member and away from the bottom member.

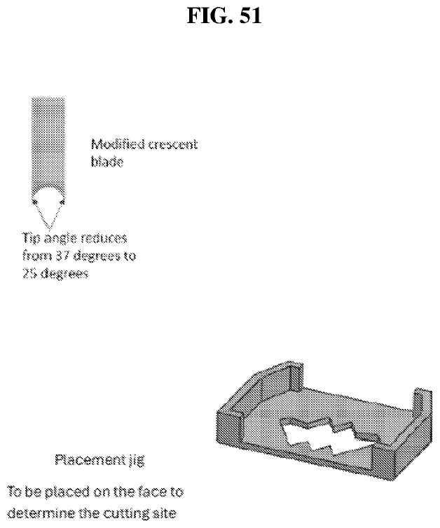

In various embodiments, the two tips may have a bevel angle of 25° (see FIG. 51).

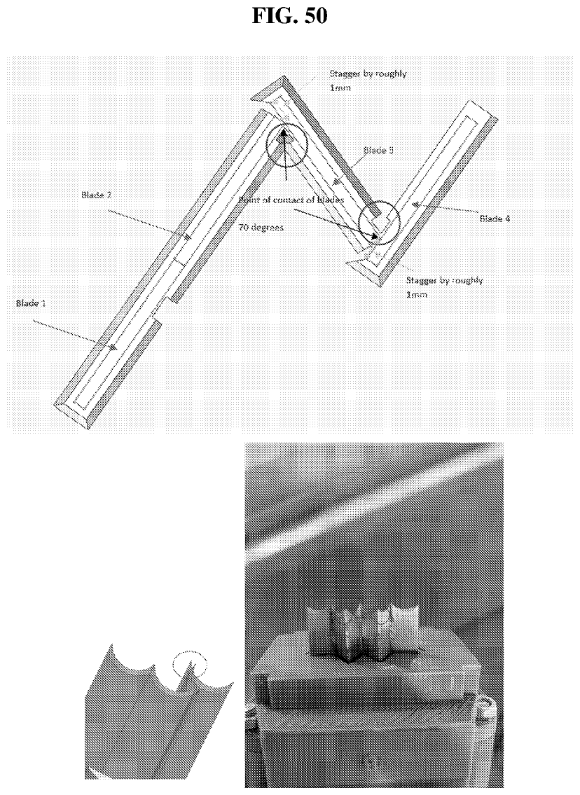

In various embodiments, the more than one blades may be staggered (see FIG. 50) to have an edge of one blade which may be in contact with another blade spaced about 1 mm away from the edge of the another blade. In other words, a pair of blades may be in contact, wherein an edge of one blade is spaced about 1 mm away from an edge of the other blade.

In summary, the present device has improved features for use in plastic surgery (e.g., scar revision) and biopsy (e.g., biopsy sample collection, skin biopsy). The present device is versatile in that it can be used for samples of various sizes, e.g., where a large skin sample is required to be removed such as in dermatology treatments.

The present device has blades with shapes and angle of the cutting tip configured to confer better cutting efficiency and less pain inflicted during the operation of the device. Also, the method of loading and releasing of the blades for penetration in skin is more steady and easier to execute. For example, the biasing member (e.g. the pin or the moveable blades) maintains the blades in a loaded position and easily operable (without shaking the device too much). As another example, the configuration of the actuation mechanism, actuating member, biasing member, striking members and blades reduces the amount of force needed to actuate the striking members and blade for penetrating the skin.

The present device can be configured for various shapes and angles of the W-plasty cut pattern. The present device can be used with various blade orientations.

The components of the present device can be constructed from various materials and with various thickness depending on needs of the device and its applications. For example, certain components may be formed of plastic or metal. As another example, certain components can be formed of optically pervious materials (e.g. transparent or translucent). As a further example, the biasing member can be formed with a thickness sufficient to withstand any load so as to avoid warping.

Optionally, the biasing member may be operably coupled to release buttons, which can be used to control the actuation mechanism, such as activating the actuating member to drive the striking members and blades for penetrating the skin.

The present device may be compatible with commercially available springs.

The present device is usable for revising an average scar having a length in the range of 2 cm to 4 cm.

In the context of various embodiments, the articles “a”, “an” and “the” as used with regard to a feature or element include a reference to one or more of the features or elements.

In the context of various embodiments, the term “about” or “approximately” as applied to a numeric value encompasses the exact value and a reasonable variance.

As used herein, the term “and/or” includes any and all combinations of one or more of the associated listed items.

Unless specified otherwise, the terms “comprising” and “comprise”, and grammatical variants thereof, are intended to represent “open” or “inclusive” language such that they include recited elements but also permit inclusion of additional, unrecited elements.

The following examples may be provided solely for illustrative purposes and may be not intended to limit the scope of the fulfillment system. The described examples illustrate specific implementations of the fulfillment system but do not encompass all possible variations. Features and aspects from different examples may be combined in various ways to suit particular applications, and modifications may be made without departing from the spirit and scope of the fulfillment system as defined by the claims.

Example 1 is a device operable to perforate a skin, the device including a housing including an upper member and a bottom member, wherein the upper member is detachably engaged to the bottom member; an actuation mechanism housed proximal to the upper member; a biasing member operably coupled to the actuation mechanism; one or more striking members; and more than one blades, each of the more than one blades operably coupled to a corresponding striking member of the one or more striking members, wherein the biasing member is operable to have the actuation mechanism activate an actuating member to drive each of the one or more striking members in a manner which renders each of the more than one blades to perforate the skin in less than 5 seconds.

In Example 2, the device of Example 1 can optionally include an intermediate member configured between and detachably engaged to the upper member and bottom member; an elevation member; and a top cover and a contact plate configured between the elevation member and the top cover, wherein the elevation member is detachably engaged to the upper member and configured between the top cover and the upper member.

In Example 3, the biasing member of Example 2 can optionally be configured between the elevation member and the top cover.

In Example 4, the biasing member of Example 2 or Example 3 an optionally include moveable plates, wherein each of the moveable plates includes a grip element for a user to push one or more of the moveable plates independently or simultaneously.

In Example 5, each of the moveable plates of Example 4 may optionally includes one or more keyholes and each of the moveable plates are position-able in a first state or in a second state, wherein in the first state the moveable plates are positioned to withhold the more than one blades from penetrating the skin, and wherein in the second state the moveable plates are positioned to have the one or more keyholes align with each corresponding striking member of the one or more striking members to be driven through by the actuating member.

In Example 6, each of the one or more keyholes of Example 5 is optionally shaped to (i) withhold the one or more striking members from being driven through by the actuating member in the first state and (ii) have each corresponding striking member of the one or more striking members passable through when driven by actuating member in the second state.

In Example 7, the actuating member of Example 5 or 6 may include a spring, wherein the spring is wrapped around each of the one or more striking members.

In Example 8, the dvice of Example 7 may be configured such that the spring is held in a compressed state between the biasing member and a top portion of the upper member and the spring is released from the compressed state when the biasing member is pushed to release the spring from the compressed state.

In Example 9, the device of Example 8 may be configured such thatthe one or more striking members are stopped at (i) a surface of the top portion and/or (ii) a surface of a bottom portion of the upper member.

In Example 10, the device of any one of Examples 2 to 9 may be configured such that the bottom member includes slits configured in line with an arrangement of the more than one blades for forming a cut pattern on the skin.

In Example 11, the device of Example 1 may further include an intermediate member configured between and detachably engaged to the upper member and bottom member.

In Example 12, the device of Example 11, the biasing member may be configured between the upper member and the intermediate member.

In Example 13, the device of Example 11 or 12, the biasing member includes moveable plates, wherein each of the moveable plates includes a grip element for a user to push or pull one or more of the moveable plates independently or simultaneously.

In Example 14, the device of Example 13, wherein each of the moveable plates includes one or more keyholes and each of the moveable plates are position-able in a first state or in a second state, wherein in the first state the moveable plates are positioned to withhold the more than one blades from penetrating the skin, and wherein in the second state the moveable plates are positioned to have the one or more keyholes align with each corresponding striking member of the one or more striking members to be driven through by the actuating member.

In Example 15, the device of Example 14, wherein the actuating member includes a spring, wherein the spring is wrapped around each of the more than one striking members.

In Example 16, the device of Example 15, wherein the spring is held in a compressed state between the biasing member and a top portion of the upper member and the spring is released from the compressed state when the biasing member is pulled or pushed to release the spring from the compressed state.

In Example 17, the device of any one of Examples 11 to 16, wherein the bottom member includes slits configured in line with an arrangement of the more than one blades for forming a cut pattern on the skin.

In Example 18, the device of any one of Examples 11 to 17, further including a contact plate configured between the upper member and the intermediate member.

In Example 19, the device of Example 1, wherein the bottom member includes slits configured in line with an arrangement of the more than one blades for forming a cut pattern on the skin; or wherein the upper member includes two opposing openings shaped to hold the biasing member abut against the actuating member; or wherein the bottom member includes two slits configured in line with an arrangement of the more than one blades for forming a cut pattern on the skin, and the upper member includes two opposing openings shaped to hold the biasing member abut against the actuating member.

In Example 20, the device of Example 19, wherein the biasing member and the two opposing openings are structurally configured to have the biasing member inserted through the upper member without the biasing member entirely passing through the two opposing openings.

In Example 21, the device of any one of Examples 1 and 19 to 20, wherein the upper member includes a window, wherein the window is configured at a portion of the upper member which is detachably engaged to the bottom member.

In Example 22, the device of any one of Examples 19 to 21, wherein the actuating member includes a spring, wherein the spring is wrapped around each of the one or more striking members.

In Example 23, the device of Example 22, wherein the spring is held in a compressed state between the biasing member and a top portion of the upper member and the spring is released from the compressed state when the biasing member is removed from the upper member, wherein the biasing member is removable in a manner to have the more than one blades perforate the skin independently or simultaneously.

In Example 24, the device of any one of Examples 10, 17 and 19, wherein the one or more striking members, the more than one blades, and the actuating member, are arranged for forming the cut pattern on the skin, wherein the cut pattern is non-linear and includes cut segments which define the cut pattern.

In Example 25, the device of Example 24, wherein the cut pattern includes a length of 2 cm to 4 cm, and/or has a W-shaped cut pattern, a V-shaped cut pattern, a S-shaped cut pattern, or any zig-zag shaped cut pattern.

In Example 26, the device of Example 25, wherein the W-shaped cut pattern includes an upper pattern and a lower pattern, wherein the upper pattern and the lower pattern each has two crosshatches which are identical to render the W-shaped cut pattern symmetrical, and wherein each of the two crosshatches of the upper pattern and each of the two crosshatches of the lower pattern are defined by an angle, wherein the angle defining each of the two crosshatches of the upper pattern is smaller than the angle defining each of the two crosshatches of the lower pattern.

In Example 27, the device of Example 26, wherein the angle defining each of the crosshatches of the lower pattern is 100° to 120°.

In Example 28, the device of any one of Examples 1 to 26, wherein each of the more than one blades is (i) positioned correspondingly to each of the one or more striking members or (ii) joined to each of the one or more striking members.

In Example 29, the device of any one of Examples 1 to 28, wherein each of the more than one blades includes a main body and an edge, wherein the edge extends from the main body by 2 mm to 5 mm and has a length of 7 mm to 9 mm, wherein the edge has a tip configured distal from the main body, wherein the tip has a bevel angle of 20° to 25°, a cutting angle of 70° to 80°, and a tip radius of 200 μm to 300 μm.

In Example 30, the device of any one of Examples 7, 15 and 22, wherein the spring renders a penetration depth of 4 mm to not more than 8 mm in the skin and the spring includes:
a spring constant of 4 N/mm or more; and/or an inner diameter of 2 mm or more; and/or an outer diameter of 5 mm or less; and/or a spring travel distance of 12 mm or more.

In Example 31, the device of any one of Examples 1 to 10 and 30, wherein the biasing member includes one moveable plate, instead of moveable plates, wherein the movable plate is coupled to a removable safety pin which is configured to restrain movement of the one moveable plate until the safety pin is removed from the device.

In Example 32, the device of Example 31, wherein the biasing member includes keyholes each positioned in alignment with a corresponding striking member so as to have all striking members pass through the keyholes simultaneously or have each of the corresponding striking member pass through the keyholes sequentially.

In Example 33, the device of Example 31 or 32, wherein the more than one blades are welded to a corresponding striking member.

In Example 34, the device of any one of Examples 31 to 33, wherein each of the more than one blades includes a blade tip having a crescent shape defined by a concave portion and two tips, wherein the two tips have a bevel angle of 37°, and wherein the blade tip has a cutting angle of 22°.

In Example 35, the device of any one of Examples 31 to 34, wherein the one or more striking members, the more than one blades, and the actuating member, are arranged for forming the cut pattern on the skin, wherein the cut pattern is non-linear and includes cut segments which define the cut pattern.

In Example 36, the device of Example 35, wherein the cut pattern includes a length of 2 cm to 4 cm, and/or has a W-shaped cut pattern.

In Example 37, the device of Example 36, wherein the W-shaped cut pattern includes an upper pattern and a lower pattern, wherein the upper pattern and the lower pattern each has three crosshatches which are identical to render the W-shaped cut pattern symmetrical, and wherein each of the three crosshatches of the upper pattern and each of the three crosshatches of the lower pattern are defined by an angle, wherein the angle defining each of the three crosshatches of the upper pattern is smaller than the angle defining two of the crosshatches of the lower pattern and is identical to one angle defining one of the crosshatches of the lower pattern.

In Example 38, the device of Example 37, wherein the angle defining each of the three crosshatches of the upper pattern is 70° and the angle defining two of the crosshatches of the lower pattern is 115°.

In Example 39, the device of any one of Examples 31 to 38, further including a lever having one end rotatably coupled to the top cover, and a portion which contacts the one moveable plate, wherein the lever is operable to have the portion push against the one moveable plate, after the removable safety pin is removed.

In Example 40, the device of any one of Examples 31 to 39, wherein a set of the more than one blades is welded to one striking member and another set of the more than one blades is welded to another striking member so as to have two striking members in the device.

In Example 41, the device of Example 40, wherein the striking member includes one extension structure longer than two other extension structures, wherein the two other extension structures are each coupled with one spring.

In Example 42, the device of Example 40 or 41, further including: a lower bushing configured between the upper member and the bottom member; and an upper bushing configured at the upper member and away from the bottom member.

In Example 43, the device of any one of Examples 40 to 42, wherein the two tips have a bevel angle of 25°.

In Example 44, the device of any one of Examples 40 to 43, wherein the more than one blades are staggered to have an edge of one blade which is in contact with another blade spaced about 1 mm away from the edge of the another blade.

While the present disclosure has been particularly shown and described with reference to specific embodiments, it should be understood by those skilled in the art that various changes in form and detail may be made therein without departing from the spirit and scope of the present disclosure as defined by the appended statements. The scope of the present disclosure is thus indicated by the appended statements and all changes which come within the meaning and range of equivalency of the statements are therefore intended to be embraced.

1. A device operable to perforate a skin, the device comprising:
a housing comprising an upper member and a bottom member, wherein the upper member is detachably engaged to the bottom member;
an actuation mechanism housed proximal to the upper member;
a biasing member operably coupled to the actuation mechanism;
one or more striking members; and
more than one blades, each of the more than one blades operably coupled to a corresponding striking member of the one or more striking members,
wherein the biasing member is operable to have the actuation mechanism activate an actuating member to drive each of the one or more striking members in a manner which renders each of the more than one blades to perforate the skin in less than 5 seconds.

2. The device of claim 1, further comprising:
an intermediate member configured between and detachably engaged to the upper member and bottom member;
an elevation member; and
a top cover and a contact plate configured between the elevation member and the top cover,
wherein the elevation member is detachably engaged to the upper member and configured between the top cover and the upper member.

3. (canceled)

4. The device of claim 2, wherein the biasing member comprises moveable plates, wherein each of the moveable plates comprises a grip element for a user to push one or more of the moveable plates independently or simultaneously.

5. The device of claim 4,
wherein each of the moveable plates comprises one or more keyholes and each of the moveable plates are position-able in a first state or in a second state,
wherein in the first state the moveable plates are positioned to withhold the more than one blades from penetrating the skin, and
wherein in the second state the moveable plates are positioned to have the one or more keyholes align with each corresponding striking member of the one or more striking members to be driven through by the actuating member.

6. (canceled)

7. The device of claim 5, wherein the actuating member comprises a spring, wherein the spring is wrapped around each of the one or more striking members.

8. The device of claim 7, wherein the spring is held in a compressed state between the biasing member and a top portion of the upper member and the spring is released from the compressed state when the biasing member is pushed to release the spring from the compressed state.

9. (canceled)

10. (canceled)

11. (canceled)

12. (canceled)

13. The device of claim 1, further comprising an intermediate member configured between and detachably engaged to the upper member and bottom member, wherein the biasing member comprises moveable plates, wherein each of the moveable plates comprises a grip element for a user to push or pull one or more of the moveable plates independently or simultaneously.

14. The device of claim 13,
wherein each of the moveable plates comprises one or more keyholes and each of the moveable plates are position-able in a first state or in a second state,
wherein in the first state the moveable plates are positioned to withhold the more than one blades from penetrating the skin, and
wherein in the second state the moveable plates are positioned to have the one or more keyholes align with each corresponding striking member of the one or more striking members to be driven through by the actuating member.

15. The device of claim 14, wherein the actuating member comprises a spring, wherein the spring is wrapped around each of the more than one striking members.

16. The device of claim 15, wherein the spring is held in a compressed state between the biasing member and a top portion of the upper member and the spring is released from the compressed state when the biasing member is pulled or pushed to release the spring from the compressed state.

17. (canceled)

18. (canceled)

19. (canceled)

20. The device of claim 1,
wherein the bottom member comprises slits configured in line with an arrangement of the more than one blades for forming a cut pattern on the skin; or
wherein the upper member comprises two opposing openings shaped to hold the biasing member abut against the actuating member; or
wherein the bottom member comprises two slits configured in line with an arrangement of the more than one blades for forming a cut pattern on the skin, and the upper member comprises two opposing openings shaped to hold the biasing member abut against the actuating member; and
wherein the biasing member and the two opposing openings are structurally configured to have the biasing member inserted through the upper member without the biasing member entirely passing through the two opposing openings.

21. (canceled)

22. (canceled)

23. (canceled)

24. (canceled)

25. The device of claim 20, wherein the cut pattern comprises a length of 2 cm to 4 cm, and/or has a W-shaped cut pattern, pattern, wherein the W-shaped cut pattern comprises an upper pattern and a lower pattern, wherein the upper pattern and the lower pattern each has two crosshatches which are identical to render the W-shaped cut pattern symmetrical, and wherein each of the two crosshatches of the upper pattern and each of the two crosshatches of the lower pattern are defined by an angle, wherein the angle defining each of the two crosshatches of the upper pattern is smaller than the angle defining each of the two crosshatches of the lower pattern.

26. (canceled)

27. The device of claim 25, wherein the angle defining each of the crosshatches of the lower pattern is 100° to 120°.

28. (canceled)

29. The device of claim 1,
wherein each of the more than one blades comprises a main body and an edge,
wherein the edge extends from the main body by 2 mm to 5 mm and has a length of 7 mm to 9 mm,
wherein the edge has a tip configured distal from the main body,
wherein the tip has a bevel angle of 20° to 25°, a cutting angle of 70° to 80°, and a tip radius of 200 μm to 300 μm.

30. (canceled)

31. The device of claim 2, wherein the biasing member comprises one moveable plate, instead of moveable plates, wherein a movable plate is coupled to a removable safety pin which is configured to restrain movement of the one moveable plate until the safety pin is removed from the device.

32. The device of claim 31, wherein the biasing member comprises keyholes each positioned in alignment with a corresponding striking member so as to have all striking members pass through the keyholes simultaneously or have each of the corresponding striking member pass through the keyholes sequentially.

33. (canceled)

34. The device of claim 31, wherein each of the more than one blades comprises a blade tip having a crescent shape defined by a concave portion and two tips, wherein the two tips have a bevel angle of 37°, and wherein the blade tip has a cutting angle of 22°.

35. The device of claim 20, wherein the one or more striking members, the more than one blades, and the actuating member, are arranged for forming the cut pattern on the skin, wherein the cut pattern is non-linear and comprises cut segments which define the cut pattern.

36. (canceled)

37. (canceled)

38. (canceled)

39. The device of claim 31, further comprising a lever having one end rotatably coupled to the top cover, and a portion which contacts the one moveable plate, wherein the lever is operable to have the portion push against the one moveable plate, after the removable safety pin is removed.

40. (canceled)

41. The device of claim 31, wherein a set of the more than one blades is welded to one striking member and another set of the more than one blades is welded to another striking member so as to have two striking members in the device; and wherein the one or another striking member comprises one extension structure longer than two other extension structures, wherein the two other extension structures are each coupled with one spring.

42. (canceled)

43. (canceled)

44. (canceled)
